(19) United States
(12) Patent Application Publication
(10) Pub. No.: US 2025/0269139 A1
(43) Pub. Date: Aug. 28, 2025

(54) FLUID GUIDE ARRANGEMENT WITH A PRESSURE MODULATOR AND VENTILATION ARRANGEMENT

(71) Applicant: Drägerwerk AG & Co. KGaA, Lübeck (DE)

(72) Inventors: Hans-Ullrich HANSMANN, Lübeck (DE); Sebastian SCHRÖTER, Lübeck (DE); Jöran BLENDERMANN, Lübeck (DE)

(21) Appl. No.: 19/059,741

(22) Filed: Feb. 21, 2025

(30) Foreign Application Priority Data
Feb. 23, 2024 (DE) 10 2024 105 138.8

(51) Int. Cl. A61M 16/20; A61M 16/00

(52) U.S. Cl. CPC A61M 16/208; A61M 16/024; A61M 2016/0027

(57) ABSTRACT

A fluid guide arrangement (100) includes a pressure modulator (15) and a valve (10), connected to an inlet fluid guide unit (3.1, 16) and an outlet fluid guide unit (17, 3.2). An inlet pressure (P2) and a control pressure (P1) determine a position of a valve body (19) relative to a valve body seat (18) at an outlet of the inlet fluid guide unit. With a gap between the valve body and the valve body seat, a fluid connection is established between the inlet fluid guide unit and the outlet fluid guide unit and is otherwise interrupted. The pressure modulator is connected to the inlet fluid guide unit and the outlet fluid guide unit and causes a pressure change (ΔP) such that the pressure at the outlet (A.15) of the pressure modulator is equal to the sum of the pressure at the inlet (E.15) and the caused pressure change.

### CROSS REFERENCE TO RELATED APPLICATIONS

This application claims the benefit of priority under 35 U.S.C. § 119 of German Application 10 2024 105 138.8, filed Feb. 23, 2024, the entire contents of which are incorporated herein by reference.

### TECHNICAL FIELD

The invention relates to a fluid guide arrangement with a pressure modulator and to a ventilation arrangement with such a fluid guide arrangement. The ventilation arrangement is capable of ventilating (artificially ventilating) a patient.

### BACKGROUND

The fluid guide arrangement according to the invention and many fluid guide arrangements known from the prior art comprise a valve with a valve body and a valve body seat. An inlet pressure (upstream pressure) and a control pressure act on the valve body. The inlet pressure and the control pressure act in opposite directions and influence the position of the valve body relative to the valve body seat and thus an outlet pressure (backpressure/downstream pressure). The outlet pressure in turn influences the volume flow out of the valve and thus the volume flow downstream of the fluid guide arrangement. With such a fluid guide arrangement, the outlet pressure and/or the volume flow can be controlled without necessarily having to control the inlet pressure.

### SUMMARY

It is an object of the invention to provide a fluid guide arrangement which makes it possible to control or regulate a setting parameter better than known fluid guide arrangements, wherein the setting parameter is a pneumatic parameter in a fluid guide unit downstream of the fluid guide arrangement.

The object is carried out by a fluid guide arrangement with features according to the invention. Advantageous embodiments are provided by this disclosure.

The fluid guide arrangement according to the invention comprises an inlet (input) fluid guide unit and an outlet (output) fluid guide unit. A “fluid guide unit” is understood to be a component which is able to guide a fluid along a trajectory and ideally prevents a relevant quantity of the fluid from leaving the trajectory. The trajectory is determined by the design, arrangement, and positioning of the component. A smooth hose, a corrugated hose, and a tube are three examples of a fluid guide unit.

Furthermore, the fluid guide arrangement comprises a valve arrangement with a first valve and optionally with at least one further valve. The first valve and the or each optional further valve each comprise a valve body and a valve body seat. If the valve arrangement comprises several valves, the valves are preferably connected in parallel. The inlet fluid guide unit comprises an outlet. The valve body of the first valve is arranged at the outlet of the inlet fluid guide unit.

The following description of the invention and the advantageous embodiments of a valve relate to the first valve. Preferably, they also apply accordingly to the or each further valve.

The valve body is movable relative to the valve body seat. Depending on the position of the valve body relative to the valve body seat, a gap occurs between the valve body and the valve body seat, or the valve body rests against the valve body seat, ideally in a fluid-tight manner. If a gap occurs between the valve body and the valve body seat, a fluid connection is established between the inlet fluid guide unit and the outlet fluid guide unit, with this fluid connection passing through the gap. If the valve body is in contact with (rests against) the valve body seat, this gap is closed, ideally completely closed, and the fluid connection is interrupted.

The valve body is located between a control chamber and the valve body seat. An inlet pressure occurs at the outlet of the inlet fluid guide unit. A control pressure occurs in the control chamber. The position of the valve body relative to the valve body seat depends on the difference between a control pressure force and an inlet pressure force. The inlet pressure force strives to move the valve body away from the valve body seat, i.e. to open the valve. The control pressure force is opposite to the inlet pressure force. The control pressure force therefore strives to move the valve body towards the valve body seat, i.e. to close the valve. The control pressure force depends on the control pressure. As a rule, the greater is the control pressure, the greater is the control pressure force. The inlet pressure force depends on the inlet pressure. As a rule, the greater is the inlet pressure, the greater is the inlet pressure force.

Furthermore, the fluid guide arrangement comprises a controllable pressure modulator with an inlet and an outlet. The inlet of the pressure modulator is connected to an inlet control fluid guide unit, the outlet is connected to an outlet control fluid guide unit. The inlet control fluid guide unit connects a branching point (junction) of the inlet fluid guide unit to the inlet of the pressure modulator. This branching point is usually an opening in the inlet fluid guide unit. A gas can flow out of the inlet fluid guide unit at the branching point and flow through the inlet control fluid guide unit to the pressure modulator. The outlet control fluid guide unit connects the outlet of the pressure modulator to the control chamber. A gas can flow from the pressure modulator through the outlet control fluid guide unit into the control chamber. The pressure at the inlet of the pressure modulator depends on the pressure at the branching point of the inlet fluid guide unit. The greater the pressure at the branching point is, the greater is the inlet pressure and the pressure at the inlet of the pressure modulator. The control pressure, i.e. the pressure in the control chamber, depends on the pressure at the outlet of the pressure modulator. The greater the pressure at the outlet of the pressure modulator is, the greater is the control pressure.

The pressure modulator can be controlled, in particular by a control unit. Depending on the control, the pressure modulator is able to cause a pressure change, namely at least a pressure reduction, optionally also a pressure increase. The pressure at the outlet of the pressure modulator is equal to the sum of the pressure at the inlet of the pressure modulator and the pressure change caused by the pressure modulator.

The fluid guide arrangement according to the invention is able to control an outlet pressure, i.e. the pressure in the outlet fluid guide unit, and/or a volume flow (volume flow rate) and/or mass flow (mass flow rate) through the outlet fluid guide unit. The volume flow is the volume per unit time that flows through a fluid guide unit, the mass flow is the mass per unit time. Because it is capable of generating an outlet pressure, the fluid guide arrangement according to the invention makes it possible in particular to control the outlet pressure and/or the volume flow and/or the mass flow, i.e. to cause the actual outlet pressure or volume flow or mass flow to follow a given course over time.

It is possible, but in many cases not necessary thanks to the invention, to control or regulate the inlet pressure or other pressure in the inlet fluid guide unit in order to generate a target (desired/required) outlet pressure, i.e. a pressure in the outlet fluid guide unit, and/or a volume flow or mass flow through the outlet fluid guide unit. In order to generate the target outlet pressure or volume flow or mass flow, it is often sufficient to generate a sufficiently high pressure and/or sufficiently high volume flow in the inlet fluid guide unit. This pressure in the inlet fluid guide unit can be constant or vary independently of the target outlet pressure or volume flow or mass flow.

One reason for this effect is as follows: Both the outlet pressure and the volume flow and mass flow through the outlet fluid guide unit depend on the area of the gap between the valve body and the valve body seat, wherein this area is available for a gas to flow through this gap. The available area in turn depends on the position of the valve body relative to the valve body seat. This relative position is influenced by the inlet pressure force and the control pressure force, usually by the difference between these two forces, and also by the configuration of the valve arrangement, which usually remains unchanged in use. The control pressure and thus the control pressure force are in turn influenced by the pressure change caused by the pressure modulator. On the other hand, the pressure change caused by the pressure modulator usually does not affect the inlet pressure and therefore the inlet pressure force. This means that the outlet pressure achieved, and the volume flow and mass flow achieved depend on the effected pressure change. In many cases, a relationship between the pressure change caused by the pressure modulator on the one hand and the resulting outlet pressure and/or the volume flow and/or the mass flow and on the other hand can be established in advance, in particular empirically, and used to control the pressure modulator. In many cases, this allows a target outlet pressure and/or a target volume flow and/or mass flow to be set relatively precisely.

According to the invention, the pressure modulator is configured to generate a pressure change. The pressure modulator is located in a control fluid guide unit, which connects the inlet fluid guide unit to the control chamber. Thanks to this connection, the pressure modulator does not need to generate the entire control pressure. Instead, the control pressure results from a superposition of the pressure change and the pressure in the inlet fluid guide unit. In other words: The pressure modulator taps (picks up) the pressure in the inlet fluid guide unit at the branching point and provides the control pressure at its outlet depending on the control. Thanks to this feature, it is sufficient if the pressure modulator is able to achieve a smaller pressure change compared to a conceivable configuration in which the pressure modulator would have to cause (create) the complete control pressure.

According to the invention, the pressure modulator is in a fluid connection with the inlet fluid guide unit via the inlet control fluid guide unit and in a fluid connection with the control chamber via the outlet control fluid guide unit. It is therefore not necessary to provide an actuator that is arranged on or in the control chamber and directly changes the control pressure there. Instead, the pressure modulator can be arranged at a distance from the control chamber and also at a distance from the inlet fluid guide unit. In many cases, this makes it easier to implement the necessary electrical and/or hydraulic and/or pneumatic connections and data connections for the pressure modulator and to make do with the available installation space.

In a preferred embodiment, the inlet fluid guide unit comprises a tapering segment with an inlet and an outlet. The outlet of the tapering segment is also the outlet of the inlet fluid guide unit at which the valve body seat is arranged. The valve body seat is therefore arranged at the outlet of the tapering segment. As mentioned above, the inlet control fluid guide unit connects a branching point in the inlet fluid guide unit to the inlet of the pressure modulator. According to this embodiment, this branching point is located in the tapering segment and there between the inlet and the outlet.

The tapering segment has an effective cross-sectional area at the inlet, at the branching point and at the outlet. The effective cross-sectional area of a fluid guide unit at a position is the size of an area at this position which is perpendicular to a flow direction through the fluid guide unit and which is available to a gas flowing through.

The feature that the tapering segment tapers from the inlet to the outlet means the following: The effective cross-sectional area at the outlet of the tapering segment is smaller than the effective cross-sectional area at its inlet, namely smaller by at least 5%, preferably by at least 10%, in particular by at least 20%. The pressure at the inlet essentially depends on the pressure generated by a fluid conveying unit (delivery unit) in the inlet fluid guide unit, minus a pressure loss due to pneumatic resistance and due to tapering (Bernoulli equation). The outlet pressure influences the inlet pressure acting on the valve body and in many cases is equal to the inlet pressure.

Note: It is known that a fluid guide unit has a pneumatic resistance. The pneumatic resistance is the quotient of the pressure loss that occurs in the fluid guide unit when a gas flows through the fluid guide unit (numerator) and the volume flow through the fluid guide unit (denominator).

In a first alternative, the effective cross-sectional area at the branching point is at least 5%, preferably at least 10%, smaller than the effective cross-sectional area at the entrance of the tapering segment. A distance occurs between the entrance and the branching point. In a second alternative, the effective cross-sectional area at the outlet is at least 5%, preferably at least 10%, smaller than the effective cross-sectional area of the tapering segment at the branching point. A distance occurs between the branching point and the outlet. These two alternatives can be combined with each other, wherein according to the combination the branching point is spaced away from (with a distance to) both the inlet and the outlet. Preferably—viewed in a direction from the inlet to the outlet—the effective cross-sectional area becomes smaller and optionally remains the same over a segment, but does not become larger.

A background for this embodiment is as follows: A typical application of the invention is that a given outlet pressure in the outlet fluid guide unit and/or a desired volume flow and/or mass flow through the outlet fluid guide unit is to be generated. In order to achieve this goal, the pressure modulator is controlled in such a way that the pressure modulator causes a pressure change given (specified) by the control. Ideally, the pressure at the inlet of the pressure modulator is equal to the pressure at the branching point minus the pressure loss due to the pneumatic resistance of the inlet control fluid guide unit. Therefore, the control pressure, i.e. the pressure in the control chamber, depends on the pressure at the branching point in the inlet fluid guide unit and on the pressure change caused. The inlet pressure, i.e. the pressure at the outlet of the inlet fluid guide unit, also depends on the pressure in the inlet fluid guide unit.

The control pressure, i.e. the pressure in the control chamber, and therefore the control pressure force depends on the pressure change, caused by the pressure modulator, and the pressure in the inlet fluid guide unit at the branching point. The pressure at the branching point depends on the effective cross-sectional area of the inlet fluid guide unit at the branching point. The inlet pressure and thus the inlet pressure force depend on the pressure at the outlet of the inlet fluid guide unit. This pressure in turn depends on the effective cross-sectional area of the inlet fluid guide unit at the outlet.

It is well known that the pressure in a fluid guide unit through which a gas flows decreases as the effective cross-sectional area decreases (Bernoulli equation). In addition, a smaller effective cross-sectional area generally leads to a higher pneumatic resistance. Therefore, for a given effective cross-sectional area at the inlet, constant volume flow and pressure in the inlet fluid guide unit, and constant effective pressure change, the lower the effective cross-sectional area at the branching point is, the lower is the control pressure, while the lower the effective cross-sectional area at the outlet is, the lower is the inlet pressure.

If the effective cross-sectional area of the inlet fluid guide unit is smaller at the branching point than at the inlet, the pressure at the branching point is lower than the pressure at the inlet of the tapering segment. The pressure at the inlet is generally equal to the pressure that a fluid conveying unit causes in the inlet fluid guide unit, minus a pressure loss due to the pneumatic resistance between the outlet of the fluid conveying unit and the inlet of the tapering segment. The lower pressure results on the one hand from the smaller cross-sectional area and on the other hand from the pressure loss caused by the pneumatic resistance of the inlet fluid guide unit on the way from the inlet to the branching point. However, if these two effective cross-sectional areas are the same size, the pressure at the branching point is only lower than the pressure at the inlet due to the pneumatic resistance.

Therefore, if the effective cross-sectional area at the branching point is smaller than the effective cross-sectional area at the inlet, the pressure at the branching point and thus the control pressure and the control pressure force are smaller by a larger amount than the pressure at the inlet and thus the inlet pressure and the inlet pressure force, compared to an embodiment in which the two effective cross-sectional areas are the same size, provided the caused pressure reduction is the same. As explained above, the control pressure force tends to close the valve, while the inlet pressure force tends to open the valve.

The embodiment just described, in which the effective cross-sectional area at the branching point is smaller than the effective cross-sectional area at the inlet, therefore has the following effect: For the same caused pressure reduction, a larger gap can be achieved between the valve body and the valve body seat compared to an embodiment in which the two effective cross-sectional areas are the same size. This in turn means that the fluid guide arrangement is able to achieve a greater outlet pressure and/or volume flow and/or mass flow for a given maximum achievable pressure reduction.

If the effective cross-sectional area of the inlet fluid guide unit is smaller at the outlet than at the branching point, the pressure at the outlet is lower than the pressure at the branching point. Again, the lower pressure results from the smaller cross-sectional area and from the pressure loss due to the pneumatic resistance. In many cases, this in turn means that even a small gap between the valve body and the valve body seat can be set relatively precisely. Therefore, in turn, a relatively small desired outlet pressure in the outlet fluid guide unit and/or relatively small volume flow and/or mass flow through the outlet fluid guide unit can also be realized relatively reliably. In some cases, this would only be possible less reliably if the effective cross-sectional area at the outlet is equal to the effective cross-sectional area at the branching point.

A further advantage of the embodiment in which the effective cross-sectional area at the outlet is smaller than the effective cross-sectional area at the branching point is as follows: If the pressure modulator does not cause a pressure change, in particular a pressure reduction, then in many cases the control pressure is also greater than the inlet pressure and then the control pressure force is also greater than the inlet pressure force. This in turn means that the valve is closed if the pressure modulator does not generate a pressure change. In many cases, a closed valve is a safe condition. The embodiment therefore often leads to a safe state when the pressure modulator is switched off or has failed.

Two alternatives for the configuration of the tapering segment have just been described. The two alternatives can be combined with each other. The two alternatives provide several design parameters in the configuration of the fluid guide arrangement according to the invention, namely the effective cross-sectional area at the inlet, the effective cross-sectional area at the branching point and the effective cross-sectional area at the outlet of the tapering segment, as well as the geometry of the tapering segment and the positioning of the branching point relative to the inlet and relative to the outlet.

The valve body seat has an effective cross-sectional area. The valve body also has an effective cross-sectional area. The effective cross-sectional area of the valve body is, for example, the area of the valve body on which the control pressure acts in order to achieve the control pressure force. The control pressure force depends on the control pressure and on the effective cross-sectional area of the valve body and is ideally equal to the product of the control pressure and the effective cross-sectional area. In other words, the effective cross-sectional area of the valve body is the quotient of the control pressure force and the control pressure that causes the control pressure force. Accordingly, the inlet pressure force depends on the inlet pressure and on the effective cross-sectional area of the valve body seat and is ideally equal to the product of the inlet pressure and the effective cross-sectional area. The effective cross-sectional area of the valve body seat is, for example, the area within a sealing ring of the valve body seat, wherein the valve body is in contact with this sealing ring when the valve is closed.

The two effective cross-sectional areas can differ from each other. Preferably, however, the effective cross-sectional area of the valve body is the same as the effective cross-sectional area of the valve body seat. If the pressure modulator does not cause a pressure change, not only is the control pressure equal to the inlet pressure, but also the control pressure force is equal to the inlet pressure force if the effective cross-sectional area is the same. In many cases, the embodiment with the matching effective cross-sectional areas achieves at least one of the following desired effects:
In particular, if the valve body being in a resting state is also in contact with the valve body seat as described below, the following is often guaranteed with even greater certainty: If the pressure modulator does not cause a pressure change, the fluid connection between the inlet fluid guide unit and the outlet fluid guide unit is interrupted. If the pressure modulator is switched off or has failed, the fluid connection is therefore interrupted, which often creates a safe condition. On the other hand, even a small pressure reduction causes an outlet pressure and thus a gap and a volume flow
The outlet pressure achieved and the volume flow and mass flow depend on the pressure change caused by the pressure modulator. With matching cross-sectional areas, the outlet pressure, volume flow, and mass flow depend relatively little, ideally not at all, on the inlet pressure. The pressure modulator can therefore be operated relatively independently of the inlet pressure.
In addition, low outlet pressures and volume flows and mass flows can also be reliably achieved by controlling the pressure modulator accordingly.

According to the invention, the valve body can be moved relative to the valve body seat. In one implementation, the valve body has a resting state. When the valve body is deflected from this resting state, the deflection causes a restoring spring force. This restoring spring force strives to return the valve body to its resting state. This restoring spring force occurs both when the valve body is moved away from the valve body seat and when the valve body is moved towards the valve body seat. When the valve body is in the resting state, no resetting spring force occurs. For example, the valve body comprises an elastic diaphragm that can be described as a spring. Or the valve body comprises a plate and a mechanical or pneumatic or hydraulic spring or a bellows or other elastic retaining element, wherein this elastic retaining element does not exert any spring force when at rest.

A design parameter in the design of the fluid guide arrangement with such a valve body is the position which the valve body assumes relative to the valve body seat when the valve body is at rest. In a preferred embodiment, the first valve is configured such that the following situation is achieved: When the control pressure force is equal to the inlet pressure force, the valve body is in contact with the valve body seat and is at rest. The or each optional further valve of the valve arrangement is preferably configured in the same way.

This embodiment has the following advantages in particular:
In many cases, it is sufficient for the pressure modulator to reduce the pressure. This pressure reduction opens the valve. The resetting spring force strives to close the valve again. It is therefore possible and often useful, but not absolutely necessary, in the embodiment just described with the resting state, for the pressure modulator to also be able to cause an increase in pressure.
Sometimes only a relatively low outlet pressure in the outlet fluid guide unit and/or a relatively low volume flow and/or mass flow should be generated through the outlet fluid guide unit, and therefore the gap between the valve body and the valve body seat should be relatively small. The area that the gap makes available for flow must often be set relatively precisely to a certain value in order to actually achieve the desired small outlet pressure or volume flow or mass flow. This is often easier if in the resting state the following situations occur: the control pressure force is equal to the inlet pressure force. The valve body is in contact with the valve body seat without force. Even a small difference between the two forces results in a gap with a desired flow area.
One conceivable alternative embodiment of the first valve is as follows: The first valve does not reach the resting state at all because the valve body exerts a restoring spring force even when it is in contact with the valve body seat. In the alternative embodiment, this restoring spring force strives to press the valve body against the valve body seat. In order to move the valve body away from the valve body seat against the restoring spring force, the pressure modulator must achieve a sufficiently large pressure reduction. With this conceivable alternative design, in some applications the pressure modulator is not able to reduce the control pressure to such an extent that the gap becomes large enough at a given inlet pressure and therefore a desired large outlet pressure and/or a desired large volume flow cannot be achieved.
If the pressure modulator is switched off or has failed, no pressure change is caused. In many cases, in the resting state embodiment, the control pressure force is equal to the inlet pressure force, the valve is in the resting state, and the fluid connection between the inlet fluid guide unit and the outlet fluid guide unit is interrupted, which is often a safe state. However, a conceivable alternative embodiment of the first valve is as follows: When the first valve is at rest, a gap occurs between the valve body and the valve body seat. In order to close this gap, the pressure modulator must cause an increase in pressure. If the pressure modulator is switched off or has failed, the pressure cannot be increased, and the valve is open. This situation is undesirable in some applications because the fluid connection is then established.
In many cases, the outlet pressure, volume flow and mass flow achieved depend relatively little on the inlet pressure in the embodiment with the resting state.

Various advantageous embodiments were described above, namely:
The effective cross-sectional area at the inlet is greater than the effective cross-sectional area at the branching point, and/or the effective cross-sectional area at the branching point is greater than the effective cross-sectional area at the outlet.
The effective cross-sectional area of the valve body seat corresponds to the effective cross-sectional area of the valve body.
If the control pressure force is equal to the inlet pressure force, the valve body is in contact with the valve body seat and is at rest (in a resting state).

At least two of these three embodiments, optionally all three embodiments, can be combined with each other. Each of the three embodiments makes it possible to achieve the following objective with greater reliability than other fluid guide arrangements: The fluid guide arrangement causes a pneumatic setting parameter of the outlet fluid guide unit, in particular the pressure in the outlet fluid guide unit (outlet pressure) or the volume flow or the mass flow, to assume a desired value depending on the pressure change. This pressure change is caused by the controlled pressure modulator. The actual dependence of the pneumatic setting parameter on the pressure change can be described by a characteristic curve (characteristic course). The aim is that this characteristic curve assumes a desired course, i.e. that the fluid guide arrangement has a desired characteristic. Furthermore, it is often desired that this characteristic curve depends relatively little, ideally not at all, on a pneumatic parameter in the inlet fluid guide unit, in particular not on the inlet pressure. This characteristic curve can be established in advance, preferably depending on tests, and can be used during operation of the fluid guide arrangement in order to control the pressure modulator.

According to the invention, the first valve comprises a valve body and a valve body seat. In one implementation, the valve body comprises a rigid plate and a flexible sheath (casing). The flexible sheath surrounds the rigid plate. When the valve body is in contact with the valve body seat, the sheath touches the valve body seat, preferably all the way around. In many cases, this configuration leads to the following desired result: Either there is no gap at all between the valve body and the valve body seat, or a completely circumferential gap occurs, which ideally has a constant thickness along the entire circumference. This effect reduces the risk of the valve body “dancing” on the valve body seat, similar to a lid on a pot of boiling water.

According to the invention, the fluid guide arrangement comprises a controllable pressure modulator which is capable of causing a pressure change. In many cases, it is sufficient for the pressure modulator to be able to bring about a pressure reduction, wherein this pressure reduction opens the valve, while a resetting spring force described above strives to close the valve. In a preferred embodiment, however, the pressure modulator is additionally able to cause an increase in pressure. In other words, the pressure change caused is greater than zero, equal to zero or less than zero, depending on the control of the pressure modulator. The pressure at the outlet of the pressure modulator can therefore be greater than, equal to or less than the pressure at the inlet of the pressure modulator. An increase in pressure caused by the pressure modulator closes the valve faster than if the valve were closed only by the resetting spring force.

In a further development of this embodiment, the pressure modulator comprises a pressure increasing modulator and a pressure reducing modulator. The pressure increasing modulator is capable of causing a pressure change greater than zero. The pressure reducing modulator is capable of causing a pressure change of less than zero. Preferably, depending on the control, the pressure increasing modulator is switched on and the pressure reducing modulator is switched off, the pressure increasing modulator is switched off and the pressure reducing modulator is switched on or both modulators are switched off. Both modulators preferably each comprise a fluid conveying unit, particularly preferably a pump, especially a piezoelectric pump. In many cases, this implementation requires relatively little installation space.

In one embodiment, the valve arrangement comprises a second valve. The second valve is connected in parallel with the first valve. The embodiment of the first valve described above preferably also applies to the second valve. The control chamber exerts a control pressure and thus a control pressure force on both the valve body of the first valve and the valve body of the second valve. The inlet fluid guide unit exerts an outlet pressure and thus an outlet pressure force on both the valve body of the first valve and the valve body of the second valve. This means that the control pressure in the control chamber and the inlet pressure in the inlet fluid guide unit act both on the valve body of the first valve and on the valve body of the second valve.

The valve body seat of the first valve has an effective cross-sectional area. The valve body seat of the second valve also has an effective cross-sectional area. In one embodiment, these two effective cross-sectional areas are the same. In another embodiment, the effective cross-sectional area of the valve body seat of the second valve is at least 10%, preferably at least 20%, smaller than the effective cross-sectional area of the valve body seat of the first valve. In many cases, the configuration with the two differently sized parallel valves makes it possible to reliably set a setting parameter of the outlet fluid guide unit to both a small value and a large value.

In one embodiment, the fluid guide arrangement comprises an inlet parameter sensor. The inlet parameter sensor comprises a measuring sensor (measuring probe) located in and/or on the inlet fluid guide unit. Preferably, a flow of fluid through the inlet fluid guide unit is capable of moving or otherwise affecting the measuring sensor. Particularly preferably, the measuring sensor is present in (engages) and operates in the inlet fluid guide unit and inevitably influences the flow of the fluid through the inlet fluid guide unit.

The inlet parameter sensor with the measuring sensor is able to measure a pneumatic parameter of the inlet fluid guide unit. The pneumatic parameter occurs in and/or at the inlet fluid guide unit. The pneumatic parameter is or comprises, in particular, the volume flow or mass flow through the inlet fluid guide unit and/or the pressure or the temperature or the humidity or the thermal conductivity in the inlet fluid guide unit or also the proportion (content, concentration, share) of a given component in a gas mixture that flows through the inlet fluid guide unit. The inlet parameter sensor uses the measuring sensor for the measurement. The measured pneumatic parameter is used, for example, to monitor the function of the fluid conveying unit and/or to detect oscillations in the inlet fluid guide unit.

Note: The formulation is used that a sensor is able to measure a physical quantity. This formulation means that the sensor is able to measure the physical quantity directly or at least one other quantity that correlates with the quantity to be measured. The or another measured quantity or the combination of the measured other quantities is therefore an indicator for the physical quantity to be measured. The measurement provides at least one value for the physical quantity sought.

The measuring sensor just mentioned is located in or on the inlet fluid guide unit and upstream of the outlet of the inlet fluid guide unit. Different positions of the measuring sensor relative to the branching point in the inlet fluid guide unit are possible. In a preferred embodiment, the branching point is located between the measuring sensor and the outlet of the inlet fluid guide unit, particularly preferably upstream of the inlet of the tapering segment. A gas mixture flowing through the inlet fluid guide unit therefore first reaches the measuring sensor and then the branching point. Part of the gas mixture is branched off at the branching point and reaches the pressure modulator, and the part of the gas mixture that is not branched off subsequently reaches the outlet and then the valve assembly. In other words: The branching point is located downstream of the measuring sensor.

This embodiment for positioning the measuring sensor relative to the branching point has the following particular advantage over other possible positioning methods: It is possible for the measured parameter or another pneumatic parameter of the inlet fluid guide unit to oscillate without these oscillations being brought about exclusively in a targeted manner by a control. In many cases, the measuring sensor engaging the inlet fluid guide unit contributes to these oscillations. According to the embodiment just described, the branching point is located downstream of the sensor. The oscillations that the measuring sensor causes, as well as oscillations that an optional component causes upstream of the sensor, therefore affect both the section of the inlet fluid guide unit located downstream of the measuring sensor and the inlet control fluid guide unit. In many cases, both the inlet pressure and the control pressure oscillate, often with an approximately similar frequency and amplitude. The oscillations therefore act on the valve body from both sides. As a result, the oscillations caused by the measuring sensor cause the valve body to oscillate to a lesser extent relative to the valve body seat compared to an embodiment in which the measuring sensor is arranged downstream of the branching point. Ideally, the inlet pressure and the control pressure even oscillate with the same frequency and the same amplitude and negligible phase shift, so that the oscillations triggered by the measuring sensor do not affect the valve body at all. The pressure change caused by the pressure modulator therefore does not need to be made dependent on these oscillations in many cases. It is often not necessary to control the pressure modulator such that the caused pressure change oscillates.

In one embodiment, the fluid guide arrangement according to the invention comprises a signal-processing controller. This controller can be used to realize a closed-loop control, i.e. a control in a control loop. The reference variable in this control is a desired time course of a pneumatic setting parameter of the outlet fluid guide unit. The pneumatic setting parameter is, in particular, the outlet pressure or the volume flow or the mass flow through the outlet fluid guide unit. A setting parameter sensor repeatedly measures the setting parameter and provides the actual time course of the setting parameter. This actual time course is the controlled variable in the control. The controller is able to control the pressure modulator with the control objective that the actual time course follows the given target time course. The pressure change caused by the controlled pressure modulator is therefore the or a manipulated variable.

In a further development of this embodiment, a characteristic curve is given in a form that can be evaluated by a computer. Preferably, this characteristic curve was determined empirically in advance and is stored. The characteristic curve describes how the setting parameter depends on the pressure change caused by the pressure modulator, i.e. on the difference between the pressure at the inlet and the pressure at the outlet of the pressure modulator.

The invention also relates to a ventilation arrangement which is suitable for artificial ventilation of a patient and comprises a fluid guide arrangement according to the invention. During ventilation, the patient is permanently or at least temporarily connected to a patient-side coupling unit or can be connected to such a unit. A breathing mask and a tube are two examples of a patient-side coupling unit that can be arranged on and/or in the patient's body.

The ventilation arrangement also comprises a fluid conveying unit and an inspiratory fluid guide unit. The fluid conveying unit is capable of expelling a breathable gas mixture which a patient can inhale. In particular, the fluid conveying unit comprises a pump, a blower, a fan, a piston-cylinder unit and/or a manual resuscitation bag. A special case of a fluid conveying unit is a reservoir that provides the breathable gas mixture under positive pressure. The inspiratory fluid conveying unit guides the expelled breathable gas mixture to the patient-side coupling unit.

A first segment of the inspiratory fluid guide unit comprises the inlet fluid guide unit and connects the fluid conveyor unit to the valve arrangement of the fluid guide arrangement. The expelled gas mixture is therefore conveyed through the first segment to the valve arrangement. The pressure in the first segment influences the inlet pressure that is applied to the first valve and to an optional further valve of the valve arrangement.

A second segment of the inspiratory fluid guide unit comprises the outlet fluid guide unit and connects the valve assembly to the patient-side coupling unit. The gas mixture, which flows through the first valve and through an optional further valve of the valve arrangement, flows through the second segment to the patient-side coupling unit. The effective cross-sectional area of the gap between the valve body and the valve body seat of the first valve and the corresponding gap of an optional further valve help to determine (set) the outlet pressure in and the volume flow and mass flow through the second segment.

A signal-processing control unit of the ventilation arrangement is able to control the pressure modulator of the fluid guide arrangement. The control of the pressure modulator causes a pressure change as described above and changes the control pressure at the first valve and optionally the control pressure at an optional further valve. By changing the control pressure at the valve or valves, at least one setting parameter assumes a given value. The given value can be variable over time. The setting parameter is a pneumatic property of the second segment. In particular, the setting parameter is a volume flow or mass flow through the second segment or a pressure in the second segment.

In particular during an artificial ventilation it is often important that the actual time course of the setting parameter follows a given target (required) time course with only slight deviation. In many cases, the invention makes it possible to achieve this goal more reliably than known ventilation arrangements.

The invention is described below by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of the ventilation arrangement of an embodiment example;

FIG. 2a is a schematic view showing the first valve in the closed position;

FIG. 2b is a schematic view showing the first valve in the open position;

FIG. 3 is a schematic sectional view showing the first valve and another valve, the pressure modulator and the two control fluid guide units;

FIG. 4 is a diagram showing four different characteristic curves that describe the dependence of the volume flow on the pressure change and refer to different positions of the diaphragm in the resting state;

FIG. 5 is a schematic view showing how a reduced cross-sectional area in the inlet fluid guide unit leads to an increasing pressure loss and thus to a lower inlet pressure;

FIG. 6a is a schematic view showing one of several possible implementations of the tapering segment of the inlet fluid guide unit;

FIG. 6b is a schematic view showing another of several possible implementations of the tapering segment of the inlet fluid guide unit;

FIG. 6c is a schematic view showing another of several possible implementations of the tapering segment of the inlet fluid guide unit;

FIG. 6d is a schematic view showing another of several possible implementations of the tapering segment of the inlet fluid guide unit;

FIG. 6e is a schematic view showing another of several possible implementations of the tapering segment of the inlet fluid guide unit;

FIG. 6f is a schematic view showing another of several possible implementations of the tapering segment of the inlet fluid guide unit;

FIG. 6g is a schematic view showing still another of several possible implementations of the tapering segment of the inlet fluid guide unit and showing the branching point located on the first segment and not on the tapering segment;

FIG. 7a is a diagram showing a dependence of the pressure upstream of the valve on the cross-sectional area;

FIG. 7b is a diagram showing a dependence of the volume flow on the pressure change and the positioning of the branching point;

FIG. 8a is a diagram showing three characteristic curves for the dependence of the volume flow on the pressure change with differing effective cross-sectional areas;

FIG. 8b is a diagram showing three characteristic curves for the dependence of the volume flow on the pressure change with matching effective cross-sectional areas;

FIG. 9a is a diagram showing three characteristic curves for the dependence of the volume flow on the pressure change with matching cross-sectional areas and with the bellows at rest with the diaphragm in contact with the valve body seat;

FIG. 9b is a diagram showing three characteristic curves for the dependence of the volume flow on the pressure change with matching cross-sectional areas and with the bellows at rest with a distance d>0 between the diaphragm and the valve body seat;

FIG. 9c is a diagram showing three characteristic curves for the dependence of the volume flow on the pressure change with matching cross-sectional areas and with the bellows compressed even when the diaphragm is in contact with the valve body seat; and

FIG. 10 is a schematic view showing an exemplary control of the volume flow through the second segment.

### DESCRIPTION OF PREFERRED EMBODIMENTS

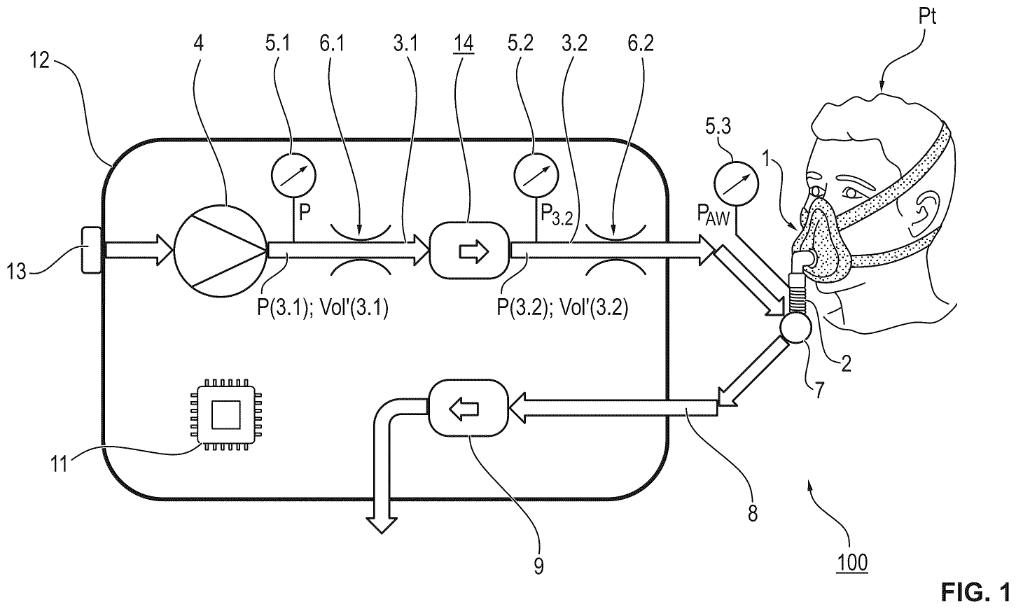

Referring to the drawings, FIG. 1 schematically shows a preferred application of the invention in a ventilation arrangement 200. A patient Pt is ventilated (artificially ventilated). A patient-side coupling unit is attached to and/or in the body of the patient Pt, in the embodiment example a breathing mask 1 on his/her face. An inspiratory fluid guide unit, for example a tube, comprises two segments 3.1 and 3.2 described below, which are connected in series, and connects a schematically shown ventilator 12 to a Y-piece 7. The first segment 3.1 connects the fluid conveying unit 4 to a valve arrangement 14, the second segment 3.2 connects the valve arrangement 14 to the Y-piece 7. The patient-side coupling unit 1 is in fluid connection with the Y-piece 7 via a fluid guide unit 2, for example via a corrugated tube. An expiratory fluid guide unit 8, for example a further tube, leads from the Y-piece 7 into the environment. An end-expiratory valve 9 is arranged in the expiratory fluid guide unit 8, which ensures that a minimum pressure is maintained in the patient's lungs Pt.

The ventilator 12 comprises a schematically shown supply (feed) connection 13 for breathing air and oxygen and optionally for compressed air and/or for an anesthetic. The ventilator 12 expels a breathable gas mixture comprising oxygen. In one implementation, the ventilator 12 is configured to generate a maximum ventilation pressure of 80 mbar at a maximum volume flow of 200 l/min at the patient-side coupling unit 1. Preferably, the ventilator 12 performs a sequence of ventilation strokes and expels a quantity of the breathing gas mixture in each ventilation stroke. The expelled gas mixture flows through the inspiratory fluid guide unit 3.1, 3.2 to the Y-piece 7 and is inhaled by the patient Pt with the aid of the patient-side coupling unit 1. The air exhaled by the patient Pt flows through the Y-piece 7 and the expiratory fluid guide unit 8 into the environment. It is also possible that a ventilation circuit is implemented, and the air exhaled by the patient Pt flows back to the ventilator 12 through the expiratory fluid guide unit 8.

A fluid conveying unit, for example a blower 4, in particular a centrifugal compressor, or a pump or a piston-cylinder unit, generates a volume flow, for example a constant volume flow over time, and a pressure, for example a constant pressure over time. The constant pressure over time is 100 mbar, for example.

It is desirable that the actual time course of a setting parameter follows a given (predetermined) target time course. The setting parameter describes a pneumatic property of the second segment 3.2 and thus at the patient-side coupling unit 1. The setting parameter is in particular the volume flow or mass flow through the second segment 3.2 or the pressure in the second segment 3.2, in particular the pressure in the airway (pressure in airway, PAW) at the patient-side coupling unit 1. For example, pressure-controlled or volume-controlled ventilation is to be performed. It is possible that two different setting parameters should each follow a predefined target time course.

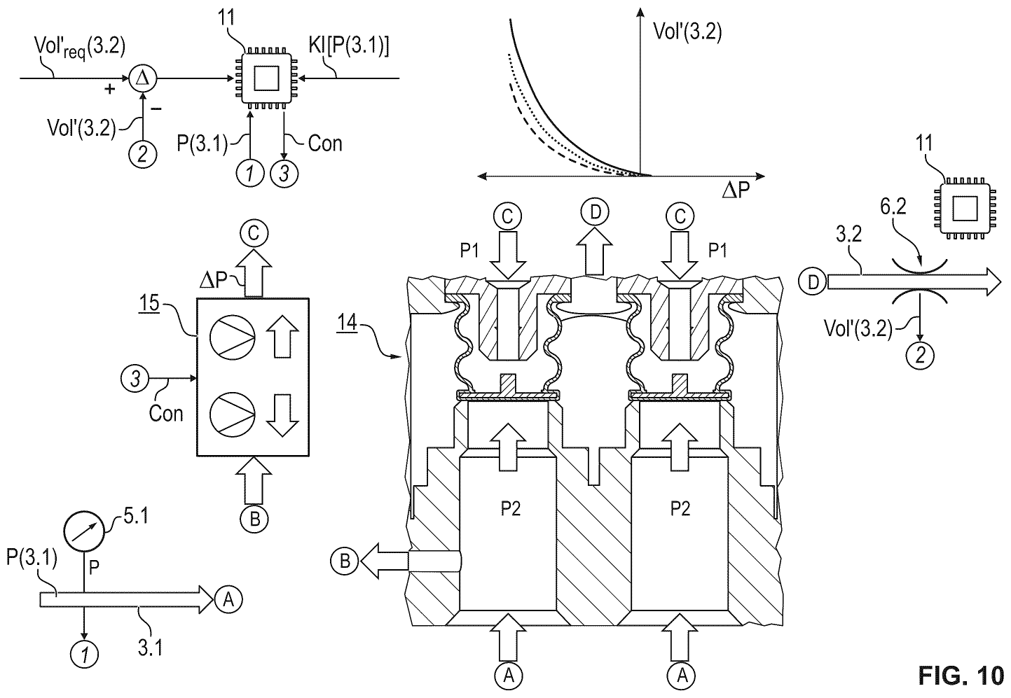

A first pressure sensor 5.1 measures the actual pressure P(3.1) in the first segment 3.1. A second pressure sensor 5.2 measures the actual pressure P(3.2) in the second segment 3.2. A third pressure sensor 5.3 measures the pressure at the airway, namely at a measuring position near the patient-side coupling unit 1. A first volume flow sensor 6.1 measures the actual volume flow Vol′(3.1) through the first segment 3.1. A second volume flow sensor 6.2 measures the actual volume flow Vol′(3.2) through the second segment 3.2. A signal-processing control unit 11 receives a signal from each of the sensors 5.1, 5.2, 5.3 and 6.1, 6.2 and controls a pressure modulator 15 described below. In the embodiment example, the control unit 11 performs automatic closed-loop control with the control objective that the actual time course of the volume flow Vol′(3.2) through the second segment 3.2 and/or that of the pressure P(3.2) in the second segment 3.2 or the time course of the airway pressure PAW or the actual time course of another setting parameter follows a given target time course, see FIG. 10.

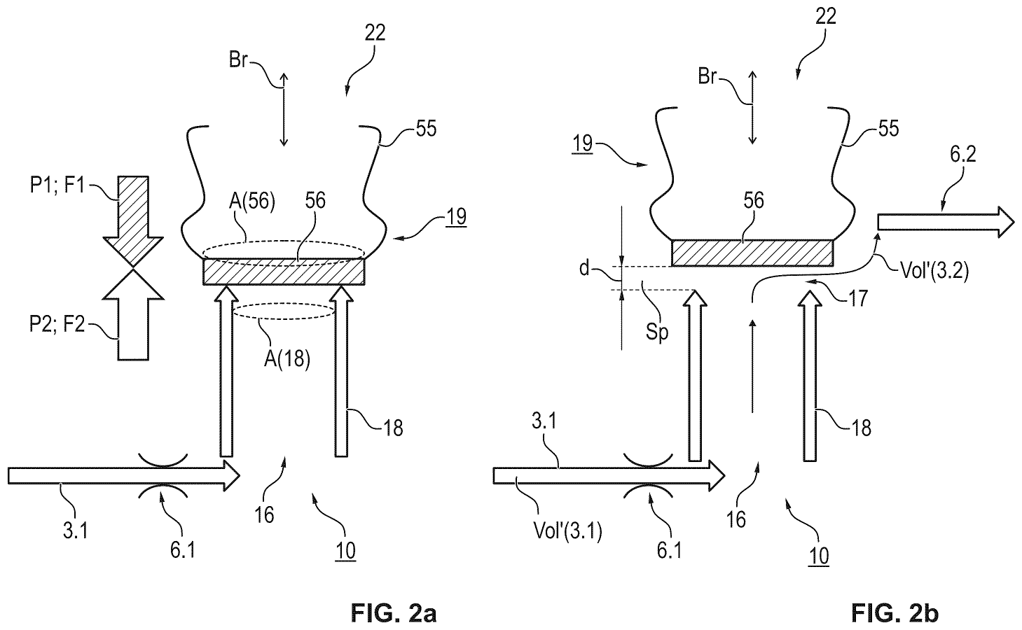

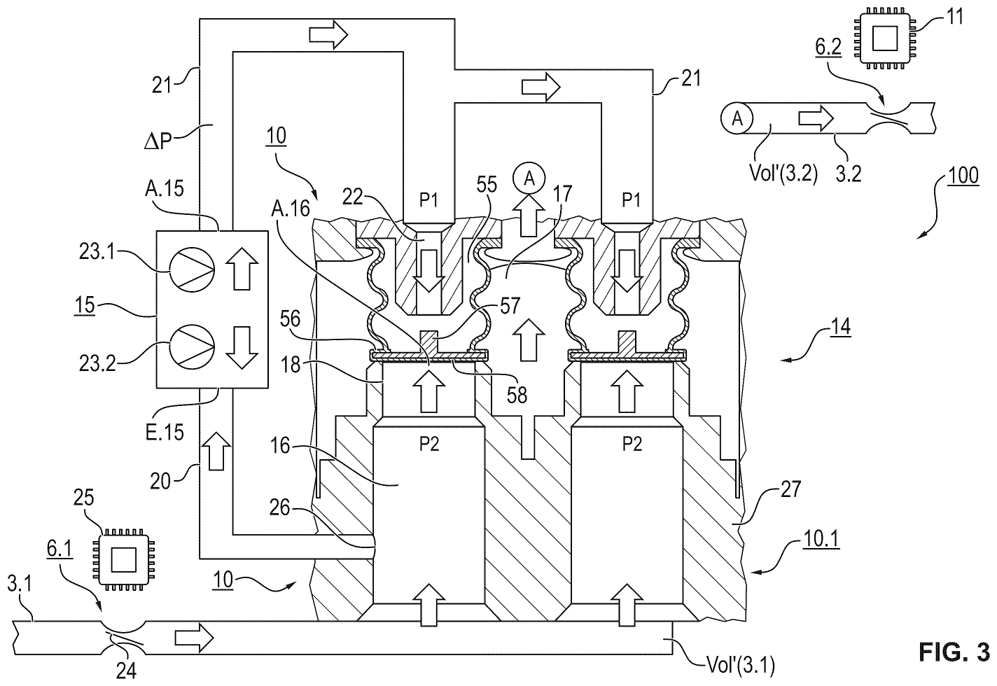

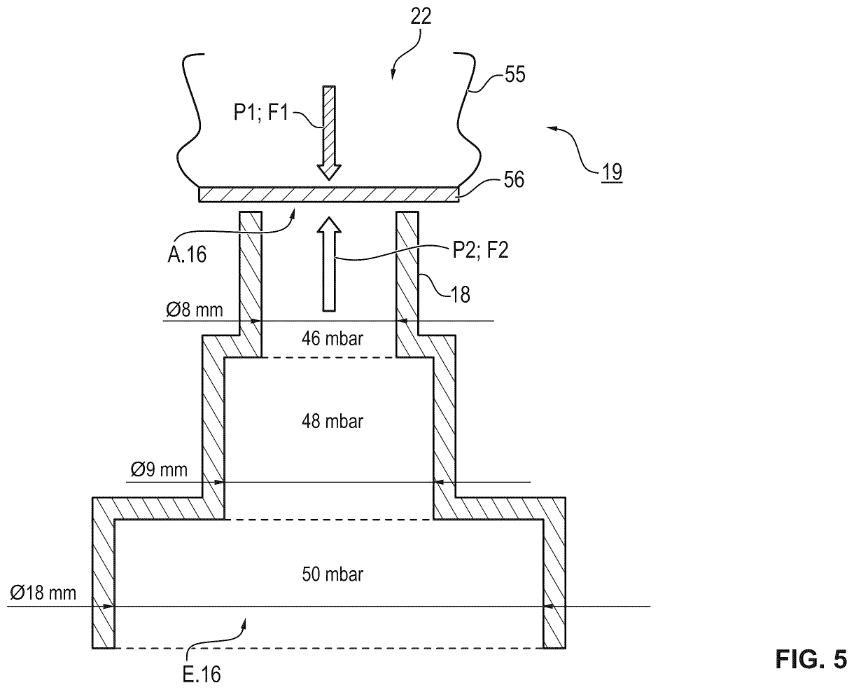

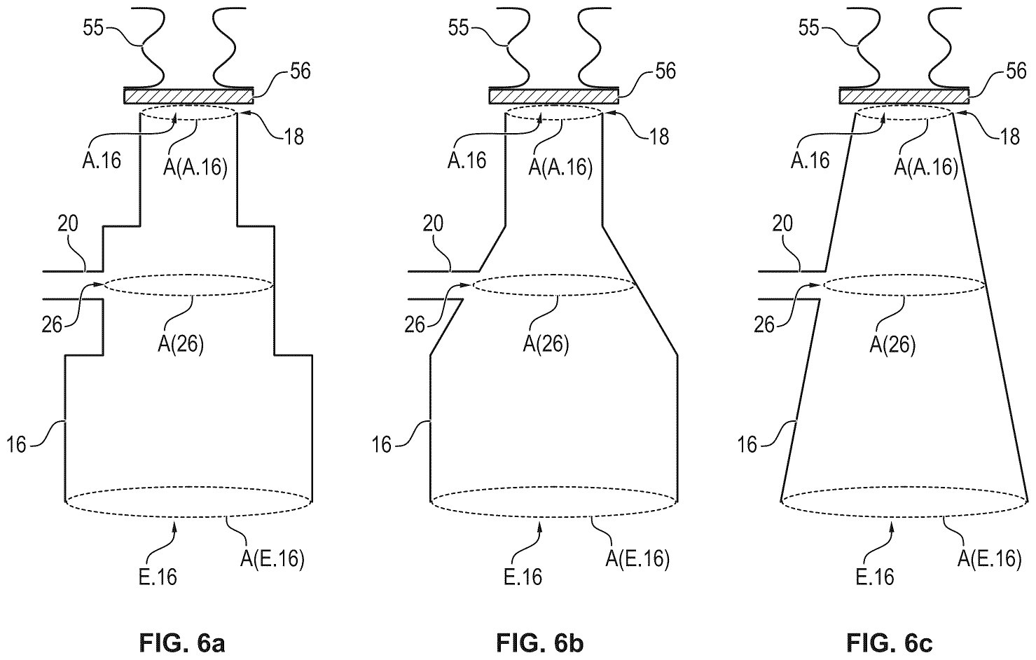

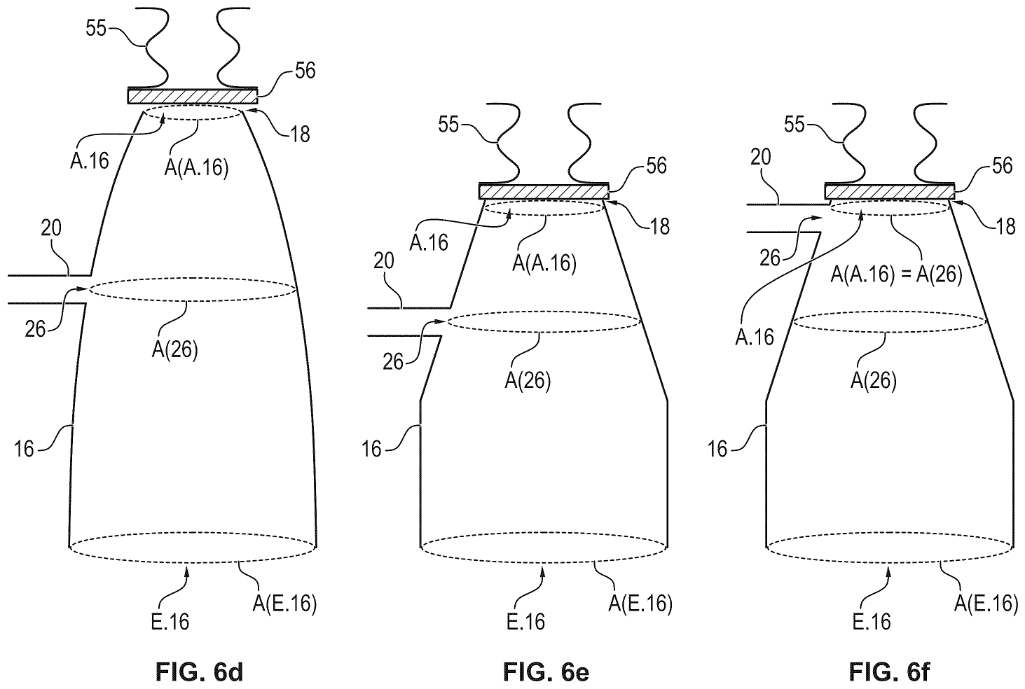

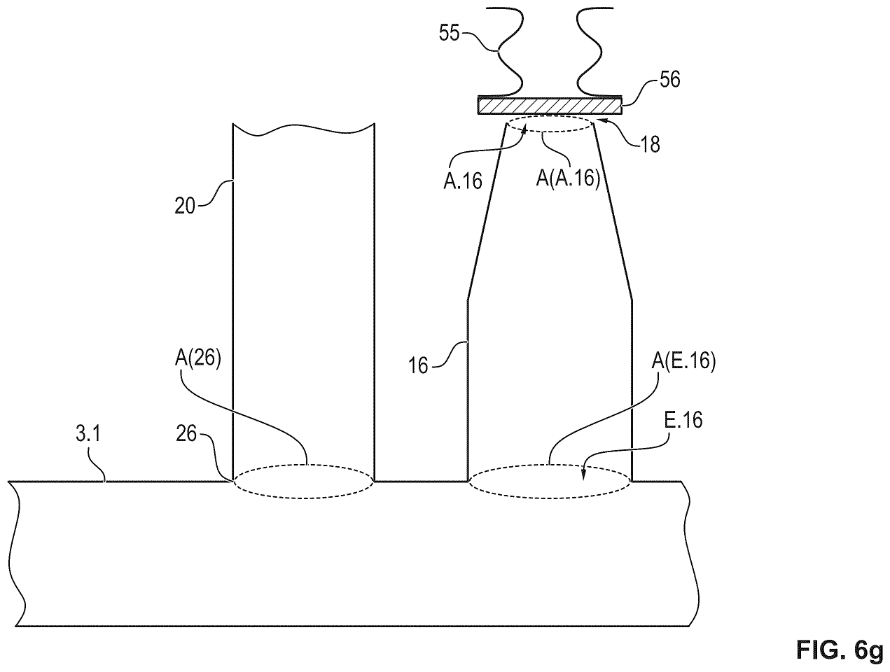

The valve arrangement 14 comprises a rigid housing 27, a first valve 10 and an optional further valve 10.1, see FIG. 3. The first valve 10 is shown schematically in FIG. 2. A fluid guide unit 16 of the valve 10 is in a fluid connection with the first segment 3.1 and thus with the fluid conveying unit 4. In the embodiment example, the fluid guide unit 16 has the shape of a tube and is referred to as inlet tube 16. The inlet tube 16 and the first segment 3.1 belong to the inlet fluid guide unit of the embodiment example. The inlet tube 16 comprises an inlet E.16 and an outlet A.16 and preferably tapers from inlet E.16 to outlet E.16, see FIG. 5 and FIG. 6. An outlet fluid guide unit 17 in the form of a further tube of the valve 10 is in a fluid connection with the second segment 3.2 and thus with the patient-side coupling unit 1. The outlet tube 17 and the second segment 3.2 belong to the outlet fluid guide unit of the embodiment example.

The first valve 10 comprises a valve body 19 and a valve body seat 18, in the example shown a circular crater 18, at the outlet A.16. In the embodiment shown, the valve body 19 comprises an elastic diaphragm 56 and an equally elastic bellows 55. In one implementation, the circular crater 18 has an inner diameter of 8 mm. A rigid plate 57 is embedded in the elastic diaphragm 56, and an elastic sheath 58 surrounds the rigid plate 57. In one implementation, the circular rigid plate 57 has a diameter of 11 mm. Preferably, the smallest diameter of the membrane 56 is larger than the largest inner diameter of the crater 18. In many cases, the membrane 56 is therefore able to close the crater 18 even if the membrane 56 is somewhat laterally offset relative to the crater 18. The rigid plate 57 is made of aluminum, for example, i.e. of a relatively light and non-magnetic material, and ideally extends in a plane that is parallel to the plane in which the round crater 18 extends. A chamber is arranged inside the membrane 56, which is filled with a gas that is under positive pressure. Thanks to the plate 57 and the chamber, the diaphragm 56 is pressed against the crater 18 in a fluid-tight manner in many situations if a control pressure force F1 described below is greater than an inlet pressure force F2. The diaphragm 56 is connected to the elastic bellows 55 in a fluid-tight manner.

The valve body 19 can—apart from often unavoidable small oscillations—move exclusively linearly relative to the valve body seat 18, up and down in the example shown. The possible relative movement is indicated in FIG. 2 by the double arrow Br. In the implementation shown, the diaphragm 56 can move linearly in two opposite directions relative to the valve body seat 18, and the bellows 55 is compressed when it is moved away from the valve body seat 18 and relaxes again when it is moved towards the valve body seat 18. The bellows 55 thus acts like a mechanical spring.

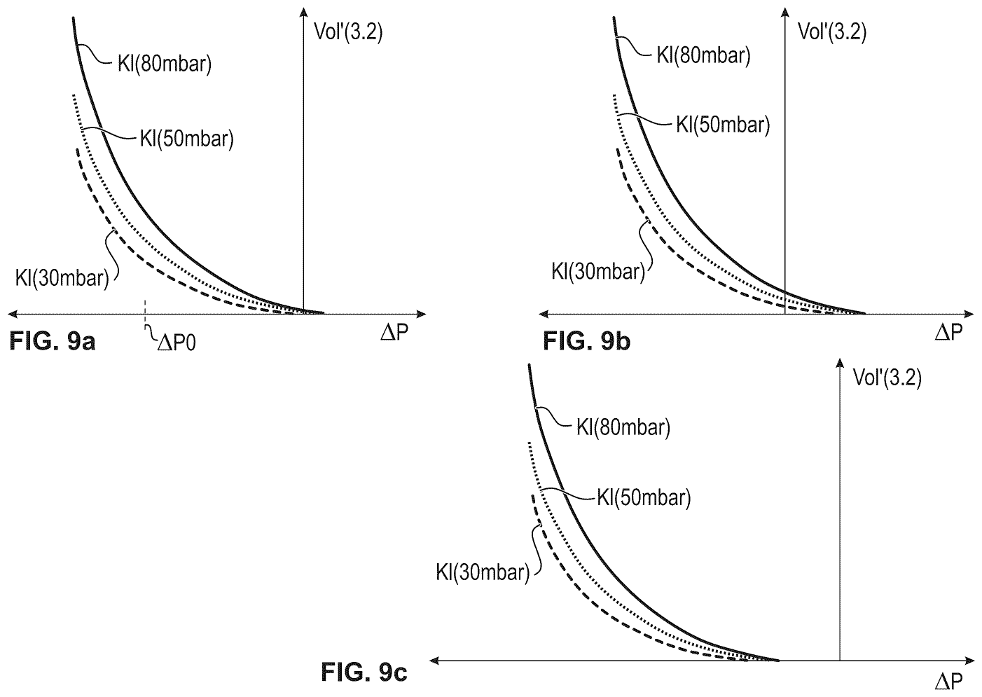

The bellows 55 is in a resting state when it is compressed or stretched again. In this resting state, the bellows 55 does not exert any spring force on the diaphragm 56. Outside the resting state, the bellows 55 exerts a restoring spring force, which strives to return (biases) the bellows 55 to the resting state. Preferably, the valve body 19 and the valve body seat 18 are positioned relative to each other as follows: When the bellows 55 is at rest, i.e. is neither stretched nor compressed and exerts no spring force, and when the control pressure force F1 is equal to the inlet pressure force F2, the diaphragm 56 lies flat against the valve body seat 18 and there is no gap between the valve body 19 and in the valve body seat 18. The diaphragm 56 is not pressed against the valve body seat 18. If the control pressure force F1 is only slightly less than the inlet pressure force F2, the gap opens. A different positioning is also possible, which is described below with reference to FIG. 9.

FIG. 2a shows the valve 10 in the closed state, in which the diaphragm 56 is in contact with the valve body seat 18. FIG. 2b shows the valve 10 in the open state, in which a circumferential gap Sp occurs between the valve body seat 18 and the diaphragm 56. A gas flows through the gap Sp. This gap Sp has the thickness (width) d. Ideally, the approximately circular gap has the same thickness d over the entire circumference. The arrow Vol′(3.2) indicates the volume flow Vol′(3.1) out of the first segment 3.1, the arrow Vol′(3.2) indicates the volume flow into the second segment 3.2. When valve 10 is closed, of course, no fluid flows through valve 10.

The bellows 55 and optionally a fluid guide unit together surround a control chamber 22. A control pressure P1 is generated in the control chamber 22. The control pressure P1 and, in one implementation, the resetting spring force of the bellows 55 described above together produce a control pressure force F1 which strives to move the valve body 19 towards the valve body seat 18 and thereby close the valve 10. In the illustration in FIG. 2, FIG. 3 and FIG. 5, the control pressure P1 and the control pressure force F1 are directed downwards.

The following applies to the control pressure force F1:

$$F 1 = P 1 * A ( 56 ) + C ( 55 ) * d . \qquad ( 1 )$$

This refers to
A(56) the area of that surface of the diaphragm 56 which is adjacent to the control chamber 22, i.e. the effective cross-sectional area of the valve body 19,
C(55) the spring constant of the bellows 55 considered as a spring and
D the ideally constant thickness (width) of the circumferential gap.

This equation (1) applies to the configuration described above, in which the valve body 19 rests in contact with the valve body seat 18 in the resting state.

An inlet pressure P2 is generated at the outlet A.16 of the inlet tube 16. The inlet pressure P2 causes an inlet pressure force F2, which strives to move the valve body 19 away from the valve body seat 18. In the illustration in FIG. 2, FIG. 3 and FIG. 5, the inlet pressure P2 and the inlet pressure force F2 are directed upwards.

The following applies to inlet pressure force F2:

$$F 2 = P 2 * A ( 18 ) . \qquad ( 2 )$$

Here, A(18) denotes the effective cross-sectional area of the valve body seat 18. In the embodiment example, A(18) is also the area of that surface of the diaphragm 56 which lies flat against the valve body seat 18 if the valve 10 is closed.

The two cross-sectional areas A(56) and A(18) are shown schematically in FIG. 2a by dashed ellipses. In this schematic example, the cross-sectional area A(56) is larger than the cross-sectional area A(18).

A resting state of the bellows 55 was described above. The bellows 55 is at rest when the bellows 55 is neither stretched nor compressed. The bellows 55 is at rest when the two opposing forces F1 and F2 are equal—unless the bellows 55 is compressed when it is in contact with the valve body seat 18 and therefore cannot assume the resting state. Preferably, the valve arrangement 14 is configured as follows: If the two forces F1 and F2 are equal, the bellows 55 is at rest. When the bellows 55 is at rest, the diaphragm 56 is in contact with the valve body seat 18 and there is no gap between the valve body 19 and the valve body seat 18, i.e. d=0 applies. In the resting state the following applies

$$P 1 * A ( 56 ) = F 1 = F 2 = P 2 * A ( 18 ) . \qquad ( 3 )$$

The configuration just described increases safety during operation for the following reason in particular: In the event of a failure, the control pressure P1 is often equal to the inlet pressure P2, and therefore the control pressure force F1 is equal to the inlet pressure force F2. The configuration just described leads to the desired result that, in the event of a failure, the fluid connection through the valve 10 is interrupted, which is a safe situation. On the other hand, the valve 10 is opened if the control pressure P2 is only slightly less than the inlet pressure P1.

FIG. 3 schematically shows an embodiment of the fluid guide arrangement 100 according to the invention. The respective flow direction of a gas is indicated by thin arrows. The following components are shown schematically:
a part of the first segment 3.1 with the first volume flow sensor 6.1,
a part of the second segment 3.2 with the second volume flow sensor 6.2,
the first valve 10 with the components already described with reference to FIG. 2,
a further valve 10.1, which has the same configuration as the first valve 10 and is arranged parallel to the first valve 10,
an inlet control fluid guide unit 20,
a pressure modulator 15 with two micropumps 23.1 and 23.2 and
an outlet control fluid guide unit 21.

In the example shown, approximately the same inlet pressure P2 and the same control pressure P1 are applied to both valves 10, 10.1.

The pressure modulator 15 comprises an inlet E.15 and an outlet A.15. The inlet control fluid guide unit 20 branches off from the inlet tube 16 at a branching point 26 and leads to the inlet E.15. The inlet control fluid guide unit 20 thus taps the pressure that is present in the inlet tube 16 at the branching point 26. The outlet control fluid guide unit 21 connects the outlet A.15 to the control chamber 22 of the first valve 10 and to a control chamber of the second valve 10.1. If the pressure modulator 15 is switched off or has failed, the control pressure P1 in the control chamber 22 is ideally equal to the pressure at the branching point 26 minus the pressure loss due to the pneumatic resistance on the path from the branching point 26 to the control chamber 22.

In the embodiment shown, the same fluid guide unit 17 functions both as an outlet tube of the first valve 10 and as an outlet tube of the second valve 10.1 and is in a fluid connection with the second segment 3.2.

FIG. 3 shows the pressure modulator 15, which determines the corresponding control pressure P1 for both valves 10, 10.1. It is also possible that each valve 10, 10.1 is assigned its own pressure modulator, so that the two control pressures can differ significantly from each other.

The fluid conveying unit 4 generates a pressure in the first segment 3.1, which is generally variable over time. The pressure in the inlet tube 16 and thus the pressure at the branching point 26 depend on the pressure generated in the first segment 3.1. The inlet E.15 of the pressure modulator 15 is connected to the inlet tube 16 via the inlet control fluid guide unit 20.

A fluid guide unit has an effective cross-sectional area. This effective cross-sectional area is the size of an area that is perpendicular to the direction of flow through the fluid guide unit and is available for flow. The effective cross-sectional area can vary over the length of the fluid guide unit. It is known that, all other things being equal, the smaller the effective cross-sectional area, the lower the pressure in a fluid guide unit (Bernoulli equation). It is known that the pneumatic resistance of a fluid guide unit generally causes a drop in pressure. When a gas flows through the flow guide unit, this pressure drop results in the pressure at the outlet of the fluid guide unit being lower than the pressure at its inlet.

The effective cross-sectional area of the inlet fluid guide unit at the branching point 26 influences the pressure in the branching point 26 and thus the pressure at the inlet E.15 of the pressure modulator 15 and thus the control pressure P1. The effective cross-sectional area of the inlet fluid guide unit at outlet A.16 influences the inlet pressure P2. The control pressure P1 and the inlet pressure P2 also depend on the pneumatic resistance of the fluid guide units involved and thus to a certain extent on the respective volume flows and the effective cross-sectional areas.

A pressure is applied to the inlet E.15 of the pressure modulator 15 that is ideally equal to the pressure at the branching point 26 minus the pressure loss due to the pneumatic resistance of the inlet control fluid guide unit 20. The control unit 11 is able to control the pressure modulator 15. At its outlet A.15, the pressure modulator 15 generates a pressure that is equal to the sum of the pressure at inlet E. 15 and a pressure change AP. The pressure modulator 15 causes the pressure change AP, wherein the pressure change AP caused depends on the control. In the embodiment example, the pressure change AP can be greater than zero, equal to zero or less than zero. The pressure at outlet A.15 can therefore be greater than, equal to or less than the pressure at inlet E.15.

The outlet control fluid guide unit 21 connects the outlet A.15 with the control chamber 22 of the first valve 10 and with the control chamber of the second valve 10.1. Therefore, the respective control pressure P1 in the control chamber 22 is equal to the pressure at the outlet A.15 minus pressure losses due to the pneumatic resistance of the outlet control fluid guide unit 21.

In the implementation shown, the pressure modulator 15 comprises a first micropump 23.1 as a pressure increasing modulator and a second micropump 23.2 as a pressure reducing modulator. The first micropump 23.1 causes a pressure change AP greater than zero (pressure increase), in one implementation by a maximum of 25 mbar. The second micropump 23.2 causes a pressure change AP less than zero (pressure reduction), in an implementation by a maximum of 25 mbar. If both micropumps 23.1, 23.2 are switched off or defective, the pressure at outlet A.15 is ideally equal to the pressure at inlet E.15. In this situation, the control pressure P2 is equal to the pressure at the branching point 26, except for possible pressure losses in the fluid guide units just described.

The first volume flow sensor 6.1 comprises a measuring sensor (measuring probe) 24 and an evaluation unit 25. The evaluation unit 25 supplies a volume flow signal, which comprises information about the measured volume flow Vol′(3.1) through the first segment 3.1. In one implementation, the sensor 24 causes a pressure drop when a gas mixture flows through the first segment 3.1. This pressure drop is an indicator of the desired volume flow Vol′(3.1). In another implementation, the evaluation unit 25 determines the difference between a pressure upstream and a pressure downstream of the sensor 24 and derives the volume flow Vol′(3.1) from the determined pressure difference. The second volume flow sensor 3.2 is preferably configured in the same way as the first volume flow sensor 3.1.

The sensor 24 engages (operates) in the first segment 3.1 and can therefore influence the volume flow Vol′(3.1) through the first segment 3.1 and therefore generate oscillations of the volume flow Vol′(3.1) through and/or oscillations of the pressure P(3.1) in the first segment 3.1. In the embodiment shown in FIG. 3, the branching point 26 is arranged downstream of the sensor 24. In other words, the branching point 26 is located between the sensor 24 and the outlet A.16 of the inlet tube 16. The oscillations caused by the sensor 24 therefore have approximately the same effect on the inlet pressure P2 and on the control pressure P1. As can be seen in FIG. 2, the control pressure P1 counteracts the inlet pressure P2. If, on the other hand, the sensor 24 were arranged downstream of the branching point 26, the oscillations would only act on the inlet pressure P2, but not on the control pressure P1, and could lead to undesired oscillation of the valve body 19 relative to the valve body seat 18 or would have to be compensated for.

As already explained, the oscillations of the sensor 24 preferably have approximately the same effect on the inlet pressure P2 and on the control pressure P1. In one embodiment, the pressure modulator 15 nevertheless compensates to a certain extent for differences between the inlet pressure P2 and the control pressure P1, which result from oscillations of the sensor 24. One reason for these differences are two different pneumatic resistances and therefore two different pressure losses in the following two fluid guide units:
on the one hand in the fluid guide unit, which leads from the branching point 26 through the inlet pipe 16 to the valve body seat 18, and
on the other hand in the fluid guide unit, which leads from the branching point 26 through the two control fluid guide units 20 and 21 and through the pressure modulator 15 to the control chamber 22.

According to this embodiment, a characteristic course (curve) is established in advance for each of these two fluid guide units, which at least approximately describes the respective pneumatic resistance as a function of the volume flow through this fluid guide unit. The first volume flow sensor 6.1 has measured the current volume flow Vol′(3.1). As a rule, the volume flow Vol′(3.1) measured by the first volume flow sensor 6.1 can also be used as the corresponding volume flow through these two fluid guide units. The measured volume flow Vol′(3.1) and the pneumatic resistance each result in a pressure loss in the two fluid guide units. The pressure modulator 15 compensates for this pressure loss. This configuration reduces possible vibrations of the valve body 19 relative to the valve body seat 18 even more.

As explained above with reference to FIG. 2, the control pressure P1 and the restoring spring force of the bellows 55 cause the control pressure force F1, see formula (1). Ideally, the control pressure P1 depends on the inlet pressure P2 as follows:

$$P 1 = P 2 + Δ P . \qquad ( 4 )$$

Here, ΔP is the pressure change (pressure modulation) caused by the pressure modulator 15, which can be less than zero, equal to zero or greater than zero in the embodiment example. In this relationship, the pressure losses due to the respective pneumatic resistance of the fluid guide units are neglected (ignored). If formula (4) is used in formula (1), the control pressure force F1:

$$F 1 = ( P 2 + Δ P ) * A ( 56 ) + C ( 55 ) * d . \qquad ( 5 )$$

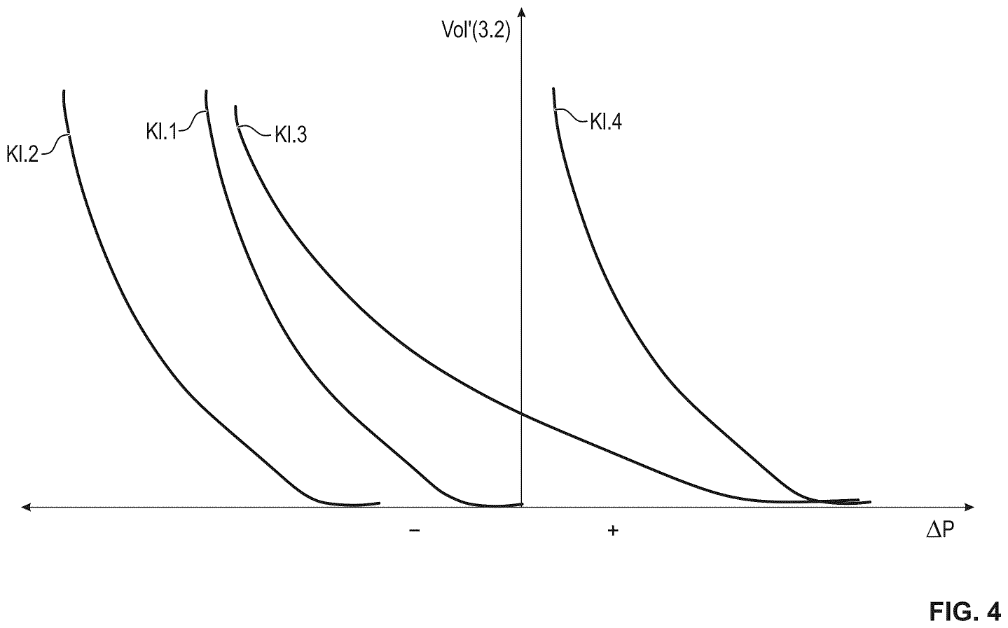

As already mentioned, the bellows 55 is elastic and strives to exert a restoring spring force when deflected from a resting state, which strives to return the bellows 55 to the resting state. The bellows 55 is at rest when the two opposing forces F1 and F2 are equal. FIG. 4 illustrates the advantage of the following situation: When the two forces F1 and F2 are equal and the bellows 55 is therefore at rest, the valve body 19 is in contact with the valve body seat 18. Because the two forces F1 and F2 coincide and the bellows 55 is at rest, there is no force that strives to move the valve body 19 relative to the valve body seat 18 in one direction or the other.

In the diagram in FIG. 4, the pressure change ΔP caused by the pressure modulator 15 is plotted on the x-axis. A positive x-value means that the control pressure P1 is greater than the inlet pressure P2, e.g. that the micropump 23.1 causes an increase in pressure and has therefore increased the pressure at outlet A.15 compared to the pressure at inlet E.15. Accordingly, a negative x-value means that the control pressure P1 is less than the inlet pressure P2, e.g. that the micropump 23.2 causes a pressure reduction and has therefore reduced the pressure at outlet A.15 compared to the pressure at inlet E.15. The resulting volume flow Vol′(3.2) through the outlet tube 17 and into the second segment 3.2 is plotted on the y-axis. For example, the volume flow sensor 6.2 measures an indicator for this resulting volume flow Vol′(3.2)

FIG. 4 shows four characteristic curves (courses) Kl.1 to Kl.4, which the inventors have obtained in internal tests. As a rule, these characteristic curves depend on the volume flow Vol′(3.1) through the first segment 3.1 and/or on the pressure P(3.1) in the first segment 3.1.

With the characteristic curve Kl.1, the situation described above is ensured, namely that when the bellows 55 is in the rest position and the two forces F1 and F2 coincide, the valve body 19 is in contact with the valve body seat 18 and no force acts on the valve body 19. This situation is created when the pressure modulator 15 does not cause any change in pressure, i.e. when ΔP=0, and when the two surfaces A(56) and A(18) are approximately the same, so that F1=F2 also applies when P1=P2. If, on the other hand, the control pressure P1 is less than the inlet pressure P2, i.e. if ΔP is less than zero, a gap Sp with a thickness d>0 occurs. The volume flow Vol′(3.2) first increases slowly and then faster as the pressure ΔP decreases. Thanks to this configuration of the characteristic curve Kl.1, a small desired (target) outlet pressure P(3.2) and/or a small desired (target) volume flow Vol′(3.2) can also be set relatively reliably.

The characteristic curve Kl.2 occurs when the bellows 55 exerts a resetting spring force even when the diaphragm 56 is in contact with the valve body seat 18. In this embodiment, the pressure modulator 15 must cause a pressure change ΔP less than zero so that the control pressure force F1 is less than the inlet pressure force F2 and the valve body 19 is thereby moved away from the valve body seat 18. In this configuration, it is often more difficult to achieve a small outlet pressure P(3.2) and/or a small volume flow Vol′(3.2). In addition, in some cases a desired high outlet pressure P(3.2) or high volume flow Vol′(3.2) can no longer be achieved because the micropump 23.2 is not able to reduce the inlet pressure P2 sufficiently.

The characteristic curves Kl.3 and Kl.4 were generated for the following situation: If the two forces F1 and F2 coincide and the bellows 55 is therefore at rest, a circumferential gap Sp occurs between the valve body 19 and the valve body seat 18, i.e. the thickness d is greater than 0. In order to close this gap Sp and thus prevent a volume flow Vol′(3.2) through the outlet tube 17, the micropump 23.1 must increase the pressure, i.e. apply a pressure change ΔP>0 to the inlet pressure P1 so that the control pressure force F1 is greater than the inlet pressure force F2 and presses the diaphragm 56 against the valve body seat 18. If the pressure modulator 15 is switched off or defective, this is not possible, so that even in the event of a failure, an often undesirable volume flow Vol′(3.2) occurs through the outlet tube 17. The two characteristic curves Kl.3 and Kl.4 differ due to the different spring constants C(55) of the bellows 55: The spring constant C(55) is greater for the characteristic curve Kl.3 than for the characteristic curve Kl.4. A further disadvantage: With large desired (target) volume flows Vol′(3.2), the micropump 23.2 is sometimes no longer able to achieve the required pressure reduction ΔP<0.

Thus, as illustrated with reference to FIG. 4, it is advantageous if the diaphragm 56 is in contact with the valve body seat 18 without force when the two forces F1 and F2 are equal and the bellows 55 is at rest. This desired state can be achieved, for example, by suspending and fastening the bellows 55 at a corresponding position in the housing 27 of the valve arrangement 14. The distance between the suspension of the bellows 55 and the valve body seat 18 is a design parameter by which the desired situation can be achieved without having to change the bellows 55 itself.

In one embodiment, a characteristic curve is determined empirically in advance for the valve arrangement 14 or for each valve 10, 10.1, wherein this characteristic curve describes the effective volume flow Vol′(3.2) through the outlet tube 17 as a function of the pressure change ΔP. As can be seen in FIG. 4 and FIG. 7a to FIG. 9c, the greater the change in pressure ΔP, the smaller the volume flow Vol′(3.2) caused. The control unit 11 applies an inverse of this characteristic curve to determine the required pressure change ΔP for a desired volume flow Vol′_{req} and controls the pressure modulator 15 in accordance with the given pressure change ΔP.

In one embodiment, which is indicated in FIG. 3, the inlet tube 16 tapers in the direction of the outlet A.16 and thus in the direction of the valve body seat 18. It has already been mentioned that a pressure loss occurs in a fluid guide unit, which depends on the pneumatic resistance of the fluid guide unit. Under otherwise constant conditions, the smaller the cross-sectional area of the fluid guide unit, the greater the pneumatic resistance. In addition, a pressure reduction according to Bernoulli's equation occurs in a tapering fluid guide unit.

FIG. 5 schematically illustrates the effect of the increasing pneumatic resistance in the tapering inlet tube 16. For clarification and illustration purposes, the inlet tube 16 tapers in two stages. In a preferred implementation, however, the inlet tube 16 tapers continuously in order to avoid undesirable turbulence.

A pressure of 50 mbar, for example, is present at the inlet E.16 of the inlet tube 16, which is generated by the fluid conveying unit 4. At inlet E.16, the inlet tube 16 has a preferably circular cross-sectional area with a diameter of 18 mm. If a volume flow Vol′(3.1) greater than zero occurs through the inlet tube 16, a pressure loss occurs which is ideally equal to the product of the volume flow Vol′(3.1) and the pneumatic resistance. FIG. 5 shows, for example, that at a diameter of 9 mm, the pressure loss results in a pressure of only 48 mbar, and at a diameter of 8 mm, a pressure of only 46 mbar. These exemplary values apply at a certain volume flow Vol′(3.1).

In one embodiment, this pressure loss is used to at least approximate a desired dependence of the resulting volume flow Vol′(3.2) on the pressure modulation ΔP. One degree of freedom (design parameter) in the configuration of the fluid guide arrangement 100 is the position of the branching point 26 in the inlet tube 16. The inlet fluid guide unit 16 has a certain effective cross-sectional area at the branching point 26. This cross-sectional area can be smaller than the cross-sectional area at the inlet E.16 and/or can be larger than the cross-sectional area at the outlet A.16.

FIG. 6 shows examples of different configurations of the tapering inlet tube 16.

-E.16 and A.16 represent the inlet and outlet of inlet tube 16 respectively,
A(E.16) is the effective cross-sectional area at inlet E.16,
A(26) is the effective cross-sectional area at branching point 26 and
A(A.16) is the effective cross-sectional area at outlet A.16.

In the examples in FIG. 6a to FIG. 6d, the following applies: A(A.16)<A(26)<A(E.16). In the example in FIG. 6e, A(A.16)=A(26)<A(E.16). In the example in FIG. 6f, the following applies: A(A.16)<A(26)<=A(E.16). The (cross-sectional area) difference is at least 5% in each case. Furthermore, in the example in FIG. 6g, the branching point is located in the first segment 3.1 and not on the tapering segment 16.

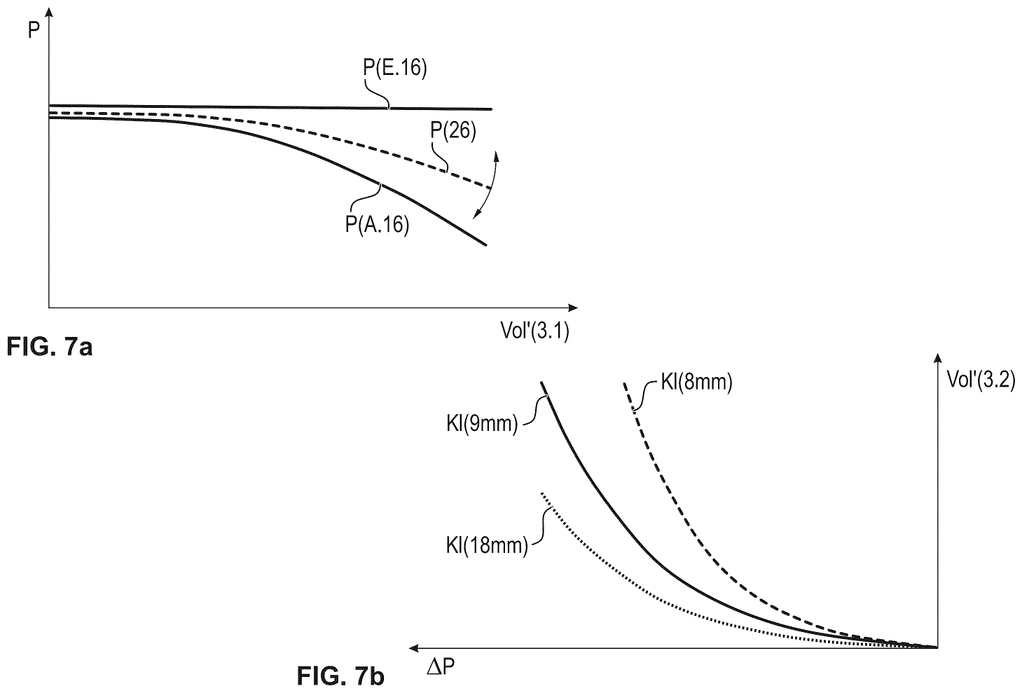

FIG. 7a shows a diagram in which the volume flow Vol′(3.1) through the first segment 3.1 is plotted on the x-axis and the resulting pressure P is plotted on the y-axis. As already mentioned, the pressure loss depends on the volume flow Vol′(3.1), the effective cross-sectional area and the pneumatic resistance. The characteristic curve P (A.16) describes the pressure at outlet A.16, which is generally equal to the inlet pressure P1. This pressure decreases relatively sharply with increasing volume flow Vol′(3.1) due to the pressure loss. The characteristic curve P (E.16) describes the pressure at inlet E.16. This pressure does not depend on the volume flow Vol′(3.1). The characteristic curve P(26) describes the pressure at the branching point 26 and thus at the inlet of the inlet control fluid guide unit 20, namely when the cross-sectional area A(26) of the inlet tube 16 at the branching point 26 is smaller than the cross-sectional area A(E.16) at the inlet E.16 and larger than the cross-sectional area A(A.16) at the outlet A.16. The double arrow indicates that the characteristic curve P(26) depends on a design parameter, namely on the effective cross-sectional area A(26) at the branching point 26 and thus on the positioning of the branching point 26 in the tapering inlet tube 16.

FIG. 7b shows a diagram in which the pressure change ΔP caused by the pressure modulator 15 is plotted on the x-axis and the resulting volume flow Vol′(3.2) through the second segment 3.2 is plotted on the y-axis, i.e. the same parameters as in FIG. 4. The three characteristic curves result from different possible positioning of the branching point 26 and thus different possible effective cross-sectional areas A(26).

The characteristic curve Kl(8 mm) results from the fact that the cross-sectional area of the inlet tube 16 has a diameter of 8 mm at the branching point 26. Correspondingly, the characteristic curves Kl(9 mm) and Kl(18 mm) result from the fact that the cross-sectional area A(26) of the inlet tube 16 has a diameter of 9 mm and 18 mm respectively at the branching point 26. The characteristic curve Kl(9 mm) leads to an advantageous dependence of the volume flow Vol′(3.2) on the pressure change ΔP. With the characteristic curve Kl(8 mm), there is a risk that the characteristic curve becomes too steep and therefore a desired low volume flow Vol′(3.2) cannot be set at all or at least not with sufficient reliability. With the characteristic curve Kl(18 mm), there is a risk that a desired high volume flow Vol′(3.2) cannot be achieved at all because the micropump 23.2 is not able to achieve a sufficiently large pressure reduction ΔP.

The advantages of an embodiment in which the bellows 55 is at rest and the diaphragm 56 is in contact with the valve body seat 18 when the two opposing forces F1 and F2 are equal were described above. In the case of force equilibrium, the relationship

$$P 1 * A ( 56 ) = F 1 = F 2 = P 2 * A ( 18 ) . \qquad ( 3 )$$

The pressure modulator 15 is configured to cause a pressure change ΔP, which affects the control pressure P1. The characteristic curves Kl.1 to Kl.4 shown in FIG. 4 apply in the event that a constant pressure P(3.1) of, for example, 50 mbar is present at the inlet E.16 of the inlet tube 16. In practice, however, this pressure P(3.1) can vary between 30 mbar and 80 mbar, for example, due to different user specifications. The pressure 3.1 has an effect on the inlet pressure P2. Another advantageous embodiment reduces the influence of the pressure P(3.1) and thus the inlet pressure P2 on the dependence of the volume flow rate Vol′(3.2) on the achieved pressure change ΔP.

Two further design parameters are the effective cross-sectional area A(56) of the diaphragm 56 and the effective cross-sectional area A(18) of the valve body seat 18, see FIG. 2. In one embodiment, the effective cross-sectional area A(56) is the quotient of the control pressure force F1 and the control pressure P1, the effective cross-sectional area A(18) is the quotient of the inlet pressure force F2 and the inlet pressure P2.

When the diaphragm 56 is in contact with the valve body seat 18, the diaphragm 56 completely covers the effective cross-sectional area A(18) of the crater 18. The effective cross-sectional area A(56) of the diaphragm 56 comes into contact with the control chamber 22.

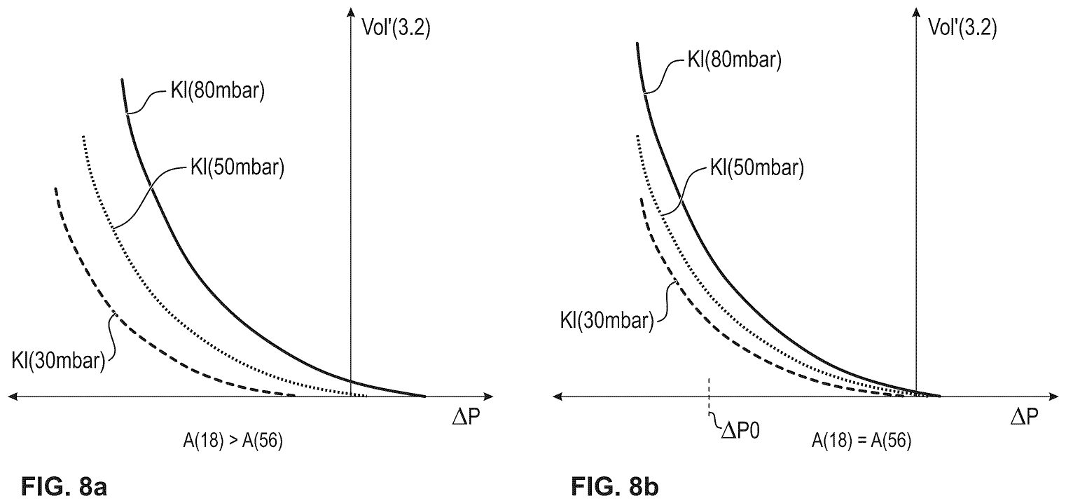

FIGS. 8a and 8b show two diagrams in which the pressure change ΔP is plotted on the x-axis and the resulting volume flow Vol′(3.2) is plotted on the y-axis. Each diagram shows three characteristic curves. The characteristic curve Kl(80 mbar) describes the dependency at a pressure P(3.1) at inlet E.16 of 80 mbar, the characteristic curve Kl(50 mbar) describes the dependency at a pressure of 50 mbar and the characteristic curve Kl(30 mbar) describes the dependency at a pressure of 30 mbar. In the diagram shown in FIG. 8a, the effective cross-sectional area A(18) of the valve body seat 18 is larger than the effective cross-sectional area A(56) of the diaphragm 56; in the diagram shown in FIG. 8b, the two effective cross-sectional areas A(18) and A(56) are the same size.

In both diagrams, the diaphragm 56 is in contact with the valve body seat 18 when the two opposing forces F1 and F2 are equal and the bellows 55 is at rest without the diaphragm 56 being pressed against the valve body seat 18. The two forces F1 and F2 depend on the pressures P1 and P2 and on the effective cross-sectional areas A(56) and A(18), see formula (3). The two effective cross-sectional areas A(56) and A(18) are predetermined by the configuration of the valve arrangement 14, and the inlet pressure force F2 is determined by the inlet pressure P2 and thus by the pressure P(3.1) in the first segment 3.1. By controlling the pressure modulator 15 accordingly, the control unit 11 is able to change the control pressure P1, namely by changing the pressure ΔP accordingly.

It is desired that no volume flow Vol′(3.2) occurs if the two opposing forces F1 and F2 are equal. Furthermore, it is desired that a relatively small pressure change ΔP already leads to a volume flow Vol′(3.2). This means that even small volume flows Vol′(3.2) can be set relatively reliably. This desired result occurs both in the diagram in FIG. 8a and in the diagram in FIG. 8b, but only at a pressure P(3.1) of 50 mbar, i.e. only for the characteristic curves Kl(50 mbar).

The diagram in FIG. 8a shows the following undesirable situations: If the two cross-sectional areas A(56) and A(18) are not the same size, but have significantly different sizes, the characteristic curve describing the volume flow Vol′(3.2) as a function of the pressure change ΔP depends considerably on the inlet pressure P2 and thus on the pressure P(3.1). At a pressure P(3.1) of 80 mbar, a gap Sp occurs between the valve body 19 and the valve body seat 18 even if the pressure modulator 15 does not cause a pressure change ΔP [characteristic curve Kl(80 mbar)]. In many cases, this results in a volume flow Vol′(3.2) even at equal pressures P1 and P2. At an inlet pressure of 30 mbar, the gap Sp only opens, and a volume flow Vol′(3.2) only starts when the pressure modulator 15 causes a sufficiently large pressure reduction ΔP [characteristic curve Kl(30 mbar)]. So before a volume flow Vol′(3.2) starts, the micropump 23.2 must first achieve a relatively large pressure reduction ΔP. In many cases, a relatively small volume flow Vol′(3.2) is relatively difficult to set with sufficient accuracy.

A conceivable remedy would be to determine a characteristic curve for each of a number of possible inlet pressures and use it during operation. If, on the other hand, the two effective cross-sectional areas A(18) and A(56) are the same size, a single characteristic curve is often sufficient to achieve the desired result, i.e. that if no pressure change ΔP is caused, no volume flow Vol′(3.2) occurs and small volume flows Vol′(3.2) can be achieved relatively reliably. This is illustrated in the diagram in FIG. 8b. In addition, the configuration in which the effective cross-sectional areas A(18) and A(56) coincide means that the valve 10 is closed when the pressure modulator 15 is switched off or has failed. In other words: If the effective cross-sectional areas A(18) and A(56) match, no volume flow Vol′(3.2) occurs if no pressure change ΔP is effected, whereas any pressure reduction ΔP<0 results in a volume flow Vol′ (3.2)>0 and wherein the smaller the effected pressure change ΔP, the greater the effected volume flow Vol′(3.2). This applies for each inlet pressure P2 and for each pressure P(3.1) in the first segment 3.1. For matching cross-sectional areas A(18) and A(56), see FIG. 8b, the following applies:

$$F 2 - F 1 = P 1 * A ( 56 ) - P 2 * A ( 18 ) = [ P 1 - P 2 ] * A ( 18 ) . \qquad ( 6 )$$

If the control pressure P1 is equal to the inlet pressure P2, the opposing forces F1 and F2 are ideally equal, regardless of the pressure P(3.1) and the volume flow Vol′(3.1).

FIGS. 9a, 9b and 9c illustrate by way of example the effect of the resting state of the bellows 55 relative to the valve body seat 18. The bellows 55 is in the resting state when the two opposing forces F1 and F2 are equal, unless the bellows 55 is compressed even with equal forces F1 and F2, and the diaphragm 56 is pressed against the valve body seat 18. The same reference signs have the same meaning in FIG. 9a, 9b, 9c as in FIGS. 8a and 8b. In all three diagrams of FIGS. 9a, 9b and 9c, the effective cross-sectional areas A(18) and A (55) correspond.

In the example in FIG. 9a, when the bellows 55 is at rest, the diaphragm 56 is in contact with the valve body seat 18. As desired, the gap Sp between the valve body 19 and the valve body seat 18 is ideally closed when the two forces F1 and F2 are equal, i.e. when no pressure change ΔP is caused, while any pressure change ΔP<0 leads to a volume flow Vol′(3.2).

In the example in FIG. 9b, when the bellows 55 is at rest, a distance d>0 occurs between the diaphragm 56 and the valve body seat 18. This results in a gap Sp between the valve body 19 and the valve body seat 18. In order to close this gap Sp, the pressure modulator 15 must apply a pressure change ΔP>0. Therefore, if the pressure modulator 15 is switched off or has failed, the gap Sp remains open, which is undesirable.

In the example in FIG. 9c, the bellows 55 is compressed even when the diaphragm 56 is in contact with the valve body seat 18. In order to move the valve body 19 away from the valve body seat 18, the pressure modulator 15 must generate a negative pressure change ΔP<0. In this arrangement, the situation may arise that the pressure reduction ΔP achievable by the pressure modulator 15 is not sufficient to achieve a desired large volume flow Vol′(3.2).

A threshold ΔP0 is shown in FIG. 8b and FIG. 9a. This is only to be understood as an example and not as a sharp threshold. The meaning of this threshold ΔP0 is as follows: The volume flow Vol′(3.2) through the gap Sp is influenced on the one hand by the effective cross-sectional area A(18) of the valve body seat 18 and on the other hand by the area A(Sp) available for flow between the valve body 19 and the valve body seat 18. The area A(Sp) available for flow is idealized as follows

$$A ( Sp ) = Umf ( 18 ) * d . \qquad ( 7 )$$

This dependency (7) applies if the gap Sp has a constant circumferential thickness d. In this case, Umf(18) is the inner circumference of the valve body seat 18, i.e. the circumference around the effective cross-sectional area A(18). If the valve body seat 18 is circular, the

$$Umf ( 18 ) = 2 * π * r ( 18 ) , \qquad ( 8 )$$

where r(18) is the radius of the valve body seat 18. If the pressure change ΔP is smaller than the threshold ΔP0, i.e. a large pressure reduction is achieved, the following effect occurs: The gap Sp is opened relatively wide. The cross-sectional area A(18) is therefore significantly smaller than the available area A(Sp), so that the volume flow Vol′(3.2) is practically only influenced by the cross-sectional area A(18) and the pressure change ΔP, but not by the available area A(Sp). In many cases, this feature makes it easier to reliably control large volume flows Vol′(3.2).

FIG. 10 shows an example of how the volume flow Vol′(3.2) is regulated through the second segment 3.2. The same reference signs have the same meaning as in the previous figures. A, B, C, D are used to illustrate fluid guide units that are not shown in FIG. 10

A target (desired) time course Vol′_{req}(3.2) of this volume flow Vol′(3.2) is given. The control objective is that the actual time course (curve) Vol′(3.2) follows the given target time course Vol′_{req}(3.2). The control unit 11 acts as a controller and receives a signal from the second volume flow sensor 6.2 that describes the controlled variable Vol′(3.2) and a signal from the first pressure sensor 5.1 that describes the pressure P(3.1) in the first segment 3.1. The control unit 11 calculates a control deviation Vol′_{req}(3.2)−Vol′(3.2). Depending on the measured pressure P(3.1), the controller 11 selects a stored characteristic curve Kl [P(3.1)]. The selected characteristic curve Kl [P(3.1)] describes the dependence of the achieved volume flow Vol′(3.2) on the pressure change ΔP at this pressure P(3.1). By evaluating this characteristic curve Kl[P(3.1)] and depending on the control deviation Vol′req (3.2)-Vol′(3.2), the controller 11 calculates a pressure change ΔP to be achieved and causes a corresponding control Con of the pressure modulator 15. The pressure modulator 15 causes the pressure change ΔP calculated by the controller 11 in accordance with the control Con, thereby reducing the control deviation.

While specific embodiments of the invention have been shown and described in detail to illustrate the application of the principles of the invention, it will be understood that the invention may be embodied otherwise without departing from such principles.

### LIST OF REFERENCE SYMBOLS

| | |
|---|---|
| 1 | patient-side coupling unit in the form of a breathing mask, connected |
| | to the fluid guide unit 2 |
| 2 | fluid guide unit, connects the Y-piece 7 with the patient-side coupling |
| | unit 1 |
| 3.1 | first segment of the inspiratory fluid guide unit, leads from the fluid |
| | conveying unit 4 to the valve arrangement 14 |
| 3.2 | second segment of the inspiratory fluid guide unit, leads from the |
| | valve arrangement 14 to the Y-piece 7 |
| 4 | Fluid conveying unit in the form of a blower, expels a gas mixture |
| | into the first segment 3.1, connected to the supply connection 13 |
| 5.1 | First pressure sensor, measures the actual pressure P(3.1) in the first |
| | segment 3.1, wherein the pressure P(3.1) is usually generated by the |
| | fluid conveying unit 4 |
| 5.2 | Second pressure sensor, measures the actual pressure P(3.2) in the |
| | second segment 3.2 |
| 5.3 | Third pressure sensor, measures the airway pressure P_{AW} |
| 6.1 | First volume flow sensor, measures an indicator of the actual volume |
| | flow Vol′(3.1) through the first segment 3.1, comprises the measuring |
| | sensor 24 and the evaluation unit 25 |
| 6.2 | Second volume flow sensor, measures an indicator of the actual |
| | volume flow Vol′(3.2) through the second segment 3.2 |
| 7 | Y-piece, connects the fluid guide units 3.2 and 8 on one side with the |
| | fluid guide unit 2 on the other side |
| 8 | Expiratory fluid guide unit, leads from the Y-piece 7 into the |
| | environment |
| 9 | End-expiratory valve in the expiratory fluid guide unit 8 |
| 10 | First valve, comprises the valve body seat 18 and the valve body 19, |
| | has the inlet tube 16 and the outlet tube 17, belongs to the valve |
| | arrangement 14 |
| 10.1 | Optional second valve, configured like the first valve 10, belongs to |
| | the valve arrangement 14 |
| 11 | Signal-processing control unit, receives and processes signals from |
| | the sensors 5.1, 5.2 and 6.1, 6.2, controls the pressure modulator 15, in |
| | one embodiment carries out a control for a setting parameter of the |
| | second segment 3.2 |
| 12 | Ventilator, comprises the fluid conveying unit 4, the control unit 11, |
| | the valve arrangement 14, the pressure modulator 15, the first segment |
| | 3.1, a part of the second segment 3.2, a part of the fluid conveying |
| | (guide) unit 8, the sensors 5.1, 5.2 and 6.1, 6.2 and the supply |
| | connection 13 |
| 13 | Supply connection of the ventilator 12, connected to the fluid |
| | conveying unit 4 |
| 14 | Valve arrangement, comprising the valve 10, the housing 27 and |
| | optionally the further valve 10.1, arranged between the segments 3.1 |
| | and 3.2 |
| 15 | Pressure modulator, comprises the micropumps 23.1, 23.2, has the |
| | inlet E.15 and the outlet A.15, processes the control Con by the |
| | control unit 11, causes the pressure change ΔP |
| 16 | Inlet tube of the first valve 10, connected to the first segment 3.1, |
| | comprises the inlet E.16 and the outlet A.16, tapers from the inlet E.16 |
| | to the outlet A.16 |
| 17 | Outlet tube of the first valve 10, connected to the second segment 3.2 |
| 18 | Valve body seat in the form of a crater, belongs to valve 10, arranged |
| | at outlet A.16, has the effective cross-sectional area A(18) |
| 19 | Valve body, comprising the diaphragm 56 and the bellows 55, belongs |
| | to the valve 10 |
| 20 | Inlet control fluid guide unit, leads from branching point 26 to inlet |
| | E.15 |
| 21 | Outlet control fluid guide unit, leads from outlet A.15 to control |
| | chamber 22 |
| 22 | Control chamber of the first valve 10, connected to the outlet A.15 via |
| | the outlet control fluid guide unit 21, partially surrounded by the |
| | bellows 55 |
| 23.1 | First micropump of the pressure modulator 15, increases the pressure |
| 23.2 | P(3.1) |
| | Second micropump of the pressure modulator 15, reduces the pressure |
| | P(3.1) |
| 24 | Sensor of the first volume flow sensor 6.1, engages in the first |
| | segment 3.1 |
| 25 | Evaluation unit of the first volume flow sensor 6.1 |
| 26 | Branching point at which the inlet control fluid guide unit 20 branches |
| | off from the inlet tube 16 of the valve 10, located between the inlet |
| | E.16 and the outlet A.16 and preferably downstream of the sensor 24 |
| 27 | Housing of the valve arrangement 14, surrounds the valves 10 and |
| | 10.1 |
| 55 | Elastic bellows, carries (supports) the diaphragm 56, fixed to the |
| | housing 27, has a resting state, exerts a restoring spring force when |
| | deflected from the resting state, has the spring constant C(55), |
| | surrounds a part of the control chamber 22 |
| 56 | Diaphragm, comprises the rigid plate 57 and the elastic sheath 58, is |
| | connected to the bellows 55, belongs to the valve body 19, has the |
| | effective cross-sectional area A(56) |
| 57 | Rigid plate 57 of the diaphragm 56, surrounded by the sheath 58, is |
| | preferably made of aluminum |
| 58 | Elastic sheath around the rigid plate 57 |
| 100 | Fluid conveying arrangement, comprises the valve arrangement 14, |
| | the control fluid guide units 20, 21 and the pressure modulator 15 |
| A.15 | Outlet of the pressure modulator 15 |
| A.16 | Outlet of the inlet tube 16 |
| A(A.16) | Effective cross-sectional area of the inlet fluid guide unit in outlet |
| | A.16 |
| A(18) | Effective cross-sectional area of the valve body seat 18, is in one |
| | embodiment equal to A(56) |
| A(26) | Effective cross-sectional area of the inlet fluid guide unit at the |
| | branching point 26 |
| A(56) | Effective cross-sectional area of the diaphragm 56, is in one |
| | embodiment equal to A(18) |
| A(A.16) | Effective cross-sectional area of the inlet fluid guide unit in inlet E.16 |
| A(Sp) | Area available for flow between the valve body 19 and the valve body |
| | seat 18 |
| Br | Directions of movement in which the valve body 19 is movable |
| | relative to the valve body seat 18 |
| Con | Control of the pressure modulator 15 by the control unit 11 |
| C(55) | Spring constant of the elastic bellows 55 |
| d | Thickness of the gap Sp |
| E.15 | Inlet of the pressure modulator 15, connected to the branching point |
| | 26 via the inlet control fluid guide unit 20 |
| E.16 | Inlet of the inlet tube 16 |
| K1.1, . . . , | Characteristic curves that describe the volume flow Vol′(3.2) through |
| K1.4 | the second segment 3.2 as a function of the pressure change ΔP |
| K1(8 mm), | Characteristic curves that describe the volume flow Vol′(3.2) through |
| K1(9 mm), | the second segment 3.2 as a function of the pressure change ΔP for a |
| K1(18 mm) | diameter of 8/9/18 mm of the inlet tube 16 at the level of the |
| | branching point 26 |
| K1(30 mbar), | Characteristic curves that describe the volume flow Vol′(3.2) through |
| K1(50 mbar), | the second segment 3.2 at an inlet pressure P(3.1) of 30/50/80 mbar |
| K1(80 mbar) | as a function of the pressure change ΔP |
| C1[P(3.1)] | Characteristic curve that describes the volume flow Vol′(3.2) through |
| | the second segment 3.2 at the inlet pressure P(3.1) as a function of the |
| | pressure change ΔP |
| P1 | Control pressure in the control chamber 22, acts on the valve body 19, |
| | is opposite to the inlet pressure P2, is the sum of the inlet pressure P2 |
| | and the pressure change ΔP as well as possible pressure losses |
| P2 | The inlet pressure in the inlet tube 16, acts on the valve body 19, |
| | opposite to the control pressure P1, caused by the fluid conveying unit |
| | 4 |
| P(3.1) | Measured pressure in the first segment 3.1 |
| P(3.2) | Measured pressure in the second segment 3.2 |
| P(A.16) | Pressure at outlet A.16 |
| P(E.16) | Pressure at inlet E.16 |
| P(26) | Pressure at branching point 26 |
| P_{AW} | Airway pressure, measured by the third pressure sensor 5.3 |
| ΔP | Pressure change caused by the pressure modulator 15 due to the |
| | control Con |
| ΔP0 | Threshold above which only the pressure change AP and the effective |
| | cross-sectional area A(18) influence the volume flow Vol′(3.2), but |
| | not the effective area A(Sp) |
| r(18) | Radius of the circular valve body seat 18 |
| Sp | Circumferential gap between the valve body 19 and the valve body |
| | seat 18, provides the area A(Sp) suitable for flow, ideally has the |
| | constant width (thickness) d |
| Umf(18) | Inner circumference of the valve body seat 18, i.e. the circumference |
| | around the effective cross-sectional area A(18) |
| Vol′(3.1) | Actual volume flow through the first segment 3.1, measured by sensor |
| | 6.1 |
| Vol′(3.2) | Actual volume flow through the second segment 3.2, measured by |
| | sensor 6.2 |
| Vol′_{req}(3.2) | Given target volume flow through the second segment 3.2, a setting |
| | parameter |

What is claimed is:

1. A fluid guide arrangement comprising:
an inlet fluid guide unit, which comprises a branching point and an outlet;
an outlet fluid guide unit;
an inlet control fluid guide unit and an outlet control fluid guide unit;
a valve arrangement, which comprises a valve, the valve comprising: a valve body seat, which is arranged at the outlet of the inlet fluid guide unit, and a valve body;
a controllable pressure modulator, which comprises an inlet and an outlet; and
a control chamber,
wherein the inlet control fluid guide unit establishes a fluid connection between the branching point of the inlet fluid guide unit and the inlet of the pressure modulator,
wherein the outlet control fluid guide unit establishes a fluid connection between the outlet of the pressure modulator and the control chamber,
wherein the valve body is movable relative to the valve body seat such that, depending on a position of the valve body relative to the valve body seat, a gap occurs between the valve body and the valve body seat, or the valve is in contact with the valve body seat,
wherein with the gap occurring, a fluid connection is established between the inlet fluid guide unit and the outlet fluid guide unit and with the valve body being in contact with the valve body seat, this fluid connection is interrupted,
wherein the valve body is located between the control chamber and the valve body seat,
wherein an inlet pressure occurs at the outlet of the inlet fluid guide unit and a control pressure occurs in the control chamber,
wherein a position of the valve body relative to the valve body seat depends on a difference between a control pressure force and an inlet pressure force,
wherein the control pressure force depends on the control pressure,
wherein the inlet pressure force depends on the inlet pressure,
wherein the control pressure force and the inlet pressure force are opposed to each other,
wherein the pressure modulator is configured to cause a pressure change depending on a control of the pressure modulator, and a pressure at the outlet of the pressure modulator is equal to a sum of the pressure at the inlet of the pressure modulator and the pressure change caused by the pressure modulator, and
wherein the greater the pressure at the outlet of the pressure modulator is, the greater is the control pressure.

2. A fluid guide arrangement according to claim 1,
wherein the inlet fluid guide unit comprises a tapering segment with an inlet and an outlet,
wherein the outlet of the inlet fluid guide unit is also the outlet of the tapering segment,
wherein the branching point is located between the inlet and the outlet of the tapering segment, and
wherein an effective cross-sectional area of the tapering segment at the branching point is at least 5% smaller than an effective cross-sectional area of the tapering segment at the inlet of the tapering segment and/or an effective cross-sectional area of the tapering segment at the outlet of the tapering segment is at least 5% smaller than an effective cross-sectional area of the tapering segment at the branching point.

3. A fluid guide arrangement according to claim 1,
wherein the valve body seat has an effective cross-sectional area, and the valve body has an effective cross-sectional area,
wherein the control pressure force depends on the control pressure and on the effective cross-sectional area of the valve body,
wherein the inlet pressure force depends on the inlet pressure and on the effective cross-sectional area of the valve body seat, and
wherein the effective cross-sectional area of the valve body and the effective cross-sectional area of the valve body seat are of equal size.

4. A fluid guide arrangement according to claim 1,
wherein the valve body has a resting state,
wherein a deflection of the valve body from the resting state causes a restoring spring force which strives to return the valve body to the resting state, and
wherein, with the control pressure force equal to the inlet pressure force, the valve body is in contact with the valve body seat and is in the resting state.

5. A fluid guide arrangement according to claim 1, wherein the pressure modulator is configured such that the pressure change caused by the pressure modulator is greater than zero, equal to zero or less than zero, depending on the control.

6. A fluid guide arrangement according to claim, 5,
wherein the pressure modulator comprises a pressure increasing modulator and a pressure reducing modulator,
wherein the pressure increasing modulator is configured to cause a pressure change greater than zero, and
wherein the pressure reducing modulator is configured to cause a pressure change of less than zero.

7. A fluid guide arrangement according to claim 1,
wherein the valve arrangement comprises the valve as a first valve and the valve arrangement further comprises a second valve,
wherein the second valve is connected in parallel with the first valve and comprises a second valve body seat and a second valve body movable relative to the second valve body seat,
wherein the second valve body is movable relative to the second valve body seat such that, depending on a position of the second valve body relative to the second valve body seat, a second valve gap occurs between the second valve body and the second valve body seat, or the second valve body rests against the second valve body seat,
wherein with the second valve gap occurring, a second fluid connection is established between the inlet fluid guide unit and the outlet fluid guide unit, and with the second valve body being in contact with the second valve body seat, the second fluid connection is interrupted,
wherein the position of the second valve body relative to the second valve body seat depends on a difference between a second control pressure force and a second inlet pressure force,
wherein the second control pressure force depends on the control pressure,
wherein the second inlet pressure force depends on the inlet pressure, and
wherein the second control pressure force and the second inlet pressure force are opposed to each other.

8. A fluid guide arrangement according to claim, 7, wherein an effective cross-sectional area of the second valve body seat is at least 10% smaller than an effective cross-sectional area of the valve body seat of the first valve.

9. A fluid guide arrangement according to claim, 7, wherein an effective cross-sectional area of the second valve body seat is at least 20% smaller than an effective cross-sectional area of the valve body seat of the first valve.

10. A fluid guide arrangement according to claim 1, further comprising an inlet parameter sensor, which comprises a measuring sensor,
wherein the measuring sensor is located in and/or on the inlet fluid guide unit,
wherein the branching point is located between the measuring sensor and the outlet of the inlet fluid guide unit, and
wherein the inlet parameter sensor is configured to measure, by using the measuring sensor, a pneumatic parameter of the inlet fluid guide unit.

11. A fluid guide arrangement according to claim 1, further comprising:
a setting parameter sensor, which is configured to measure a pneumatic setting parameter of the outlet fluid guide unit; and
a control unit configured as a controller,
wherein the controller is configured to control the pressure modulator depending on a signal from the setting parameter sensor with a control objective that an actual time course of the setting parameter follows a given time course.

12. A fluid guide arrangement according to claim 11,
wherein the controller is configured to control the pressure modulator as a function of a given characteristic curve that can be evaluated by a computer,
wherein the characteristic curve describes a dependance of the pneumatic setting parameter on the pressure change caused by the pressure modulator.

13. A ventilation arrangement for artificial ventilation of a patient, wherein during ventilation the patient is connected or can be connected at least temporarily to a patient-side coupling unit, the ventilation arrangement comprising:
a fluid conveying unit;
an inspiratory fluid guide unit with a first segment and a second segment;
a fluid guide arrangement, the fluid guide arrangement comprising: an inlet fluid guide unit, which comprises a branching point and an outlet; an outlet fluid guide unit; an inlet control fluid guide unit and an outlet control fluid guide unit; a valve arrangement, which comprises a valve, the valve comprising: a valve body seat, which is arranged at the outlet of the inlet fluid guide unit, and a valve body; a controllable pressure modulator, which comprises an inlet and an outlet; and a control chamber, wherein the inlet control fluid guide unit establishes a fluid connection between the branching point of the inlet fluid guide unit and the inlet of the pressure modulator, wherein the outlet control fluid guide unit establishes a fluid connection between the outlet of the pressure modulator and the control chamber, wherein the valve body is movable relative to the valve body seat such that, depending on a position of the valve body relative to the valve body seat, a gap occurs between the valve body and the valve body seat, or the valve body is in contact with the valve body seat, wherein with the gap occurring, a fluid connection is established between the inlet fluid guide unit and the outlet fluid guide unit and with the valve body being in contact with the valve body seat, this fluid connection is interrupted, wherein the valve body is located between the control chamber and the valve body seat, wherein an inlet pressure occurs at the outlet of the inlet fluid guide unit and a control pressure occurs in the control chamber, wherein a position of the valve body relative to the valve body seat depends on a difference between a control pressure force and an inlet pressure force, wherein the control pressure force depends on the control pressure, wherein the inlet pressure force depends on the inlet pressure, wherein the control pressure force and the inlet pressure force are opposed to each other, wherein the pressure modulator is configured to cause a pressure change depending on a control of the pressure modulator, and a pressure at the outlet of the pressure modulator is equal to a sum of the pressure at the inlet of the pressure modulator and the pressure change caused by the pressure modulator, and wherein the greater the pressure at the outlet of the pressure modulator is, the greater is the control pressure; and
a signal-processing control unit,
wherein the first segment of the inspiratory fluid guide unit comprises the inlet fluid guide unit and connects the fluid conveying unit to the valve arrangement,
wherein the second segment of the inspiratory fluid guide unit comprises the outlet fluid guide unit and connects the valve arrangement to the patient-side coupling unit,
wherein the fluid conveying unit is adapted to generate a flow of a gas through the first segment to the valve assembly and to cause the inlet pressure at the first valve,
wherein the control unit is configured to control the pressure modulator with the control objective that the control of the pressure modulator changes the control pressure such that at least one setting parameter assumes a predefined value, and
wherein the setting parameter is a pneumatic property of the second segment.

14. A ventilation arrangement according to claim 13, wherein the pneumatic property of the second segment comprises a volume flow or mass flow through the second segment and/or a pressure in the second segment.

15. A ventilation arrangement according to claim 13,
wherein the inlet fluid guide unit comprises a tapering segment with an inlet and an outlet,
wherein the outlet of the inlet fluid guide unit is also the outlet of the tapering segment,
wherein the branching point is located between the inlet and the outlet of the tapering segment, and
wherein an effective cross-sectional area of the tapering segment at the branching point is at least 5% smaller than an effective cross-sectional area of the tapering segment at the inlet of the tapering segment and/or an effective cross-sectional area of the tapering segment at the outlet of the tapering segment is at least 5% smaller than an effective cross-sectional area of the tapering segment at the branching point.

16. A ventilation arrangement according to claim 15,
wherein the valve body seat has an effective cross-sectional area, and the valve body has an effective cross-sectional area,
wherein the control pressure force depends on the control pressure and on the effective cross-sectional area of the valve body,
wherein the inlet pressure force depends on the inlet pressure and on the effective cross-sectional area of the valve body seat, and
wherein the effective cross-sectional area of the valve body and the effective cross-sectional area of the valve body seat are of equal size.

17. A ventilation arrangement according to claim 13,
wherein the valve body has a resting state,
wherein a deflection of the valve body from the resting state causes a restoring spring force which strives to return the valve body to the resting state, and
wherein with the control pressure force being equal to the inlet pressure force, the valve body is in contact with the valve body seat and is in the resting state.

18. A ventilation arrangement according to claim 13,
wherein the pressure modulator is configured such that the pressure change caused by the pressure modulator is greater than zero, equal to zero or less than zero, depending on the control,
wherein the pressure modulator comprises a pressure increasing modulator and a pressure reducing modulator,
wherein the pressure increasing modulator is configured to cause a pressure change greater than zero, and
wherein the pressure reducing modulator is configured to cause a pressure change of less than zero.

19. A ventilation arrangement according to claim 13,
wherein the valve arrangement comprises the valve as a first valve and the valve arrangement further comprises a second valve,
wherein the second valve is connected in parallel with the first valve and comprises a second valve body seat and a second valve body movable relative to the second valve body seat, wherein the second valve body is movable relative to the second valve body seat such that, depending on a position of the second valve body relative to the second valve body seat, a second valve gap occurs between the second valve body and the second valve body seat, or the second valve body rests against the second valve body seat,
wherein with the second valve gap occurring, a second fluid connection is established between the inlet fluid guide unit and the outlet fluid guide unit, and with the second valve body being in contact with the second valve body seat, the second fluid connection is interrupted,
wherein the position of the second valve body relative to the second valve body seat depends on a difference between a second control pressure force and a second inlet pressure force,
wherein the second control pressure force depends on the control pressure,
wherein the second inlet pressure force depends on the inlet pressure, and
wherein the second control pressure force and the second inlet pressure force are opposed to each other.

20. A ventilation arrangement according to claim 13, further comprising a setting parameter sensor,
wherein the setting parameter sensor is configured to measure a pneumatic setting parameter of the outlet fluid guide unit,
wherein the controller is configured to control the pressure modulator depending on a signal from the setting parameter sensor with a control objective that an actual time course of the setting parameter follows a given time course.
